(19) United States
(12) Patent Application Publication
(10) Pub. No.: US 2006/0287318 A1
(43) Pub. Date: Dec. 21, 2006

(54) Method for treating glaucoma III B

(76) Inventors: Dilip Wagle, New York, NY (US); Martin Gall, Morristown, NJ (US); Stanley C. Bell, Narberth, PA (US); Edmond J. LaVoie, Princeton Junction, NJ (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/295,213

(22) Filed: Dec. 5, 2005

Related U.S. Application Data
(63) Continuation of application No. 10/038,114, filed on Dec. 31, 2001, now abandoned.
(60) Provisional application No. 60/259,429, filed on Dec. 29, 2000.
(60) Provisional application No. 60/296,317, filed on Jun. 6, 2001.

(51) Int. Cl. A61K 31/53; A61K 31/505; A61K 31/50; A61K 31/4965; A61K 31/44

(52) U.S. Cl. 514/241; 514/247; 514/252.1; 514/256; 514/358

(57) ABSTRACT

Provided, among other things, is a method of decreasing intraocular pressure or improving ocular accommodation in an animal, including a human, comprising administering an intraocular pressure decreasing or ocular accommodation improving amount of a compound of the formula (1): Y-Ar⊕•X⁻

The present application is a continuation of U.S. Ser. No. 10/038,114, filed Dec. 31, 2001, which claims the benefit of and priority to U.S. Ser. No. 60/259,429, filed Dec. 29, 2000 and U.S. Ser. No. 60/296,317, filed Jun. 6, 2001. Each of these applications is incorporated herein by reference in their entirety.

The present invention relates to methods for treating glaucoma or improving accommodation (i.e. the process by which the eye adjusts for vision at different distances), and to compounds and compositions for use in such treating. In one aspect, the present invention relates to a method of decreasing the intraocular pressure caused by glaucoma.

Diabetes is the major determinant to the development of visual disability and blindness in parts of the world unencumbered by causes related to malnutrition or infectious diseases. Retinopathy is the leading cause of blindness in diabetics and is a progressive, degenerative disease. Of the many risk factors believed to be associated with diabetic retinopathy, the level of glucose in the plasma has been widely investigated. It is well accepted that a lower incidence of retinopathy is associated with decreased plasma levels of glucose.

Ophthalmologic disorders in diabetes include opacification and glaucoma. As the occurrence of these indications is correlated with the persistent hyperglycemia of the disease. Although the incidence of glaucoma is significant in diabetic populations, glaucoma affects a substantial portion of the general aging population as well.

Primary open angle glaucoma occurs in approximately 4% of diabetics compared to 1.8% of the general population. The reasons for the increase in intraocular pressure that is observed in this disorder are not completely understood. The increase in intraocular pressure that characterizes glaucoma is likely caused by an impairment in the drainage of fluid from the eye at the trabecular meshwork since trabeculectomy restores, at least for a period of time, normal intraocular pressures. The origin of this impairment to fluid movement is currently unknown but may be related to a physical obstruction or restriction to movement of proteins that make up a sieving system in the trabecular meshwork. The trabecular meshwork functions as a sieving system that maintains a restricted flow of intraocular fluid from the eye. The result of excess restriction of this flow is a back pressure that causes increased intraocular pressure.

Replacement of the trabecular meshwork (trabeculectomy) remains an established surgical procedure for improving the filtering of intraocular fluid and for overall reduction of intraocular pressure. This remedy is invasive and of limited effectiveness, since pressure elevation frequently recurs after the procedures.

Current chronic pharmaceutical therapies impose a measure of risk on an already medically compromised patient population. The use of topical B-blockers may affect underlying cardiovascular disease, and carbonic anhydrase inhibitors (e.g. Diamox™) may cause metabolic acidosis. The use of pressure-lowering drugs will be affected by the state of renal disease in compromised elderly and diabetic patients. The drawbacks associated with current pharmaceutical therapies highlight an unmet medical need for a chronic pharmaceutical intervention that is distinct in mechanism of action from current therapies.

New strategies for pharmaceutical intervention in the treatment of glaucoma based upon new mechanisms of action need to be identified. In addition, pharmaceutical agents that decrease the intraocular pressure associated with glaucoma are needed. Also, the methods of improving accommodation provided by the invention allow one to avoid costly and burdensome optical solutions, such as the use of separate reading glasses or glasses with bifocal lenses.

### SUMMARY OF THE INVENTION

In one embodiment, the invention relates to a method of treating or preventing or ameliorating glaucoma, decreasing intraocular pressure or improving or ameliorating ocular accommodation in an animal, including a human, comprising administering an intraocular pressure decreasing or accommodation improving amount of a compound of formula (I):
Y—Ar^{⊕}•X⁻ (I)
wherein Ar is a five or six membered heteroaryl ring having a first ring nitrogen and optionally second or third ring nitrogens, with the remaining ring atoms being carbon, oxygen, or sulfur, provided the first nitrogen of Ar is a quaternary nitrogen and Ar is not thiazolium, oxazolium or imidazolium. Y and other substituents on Ar are defined below.

### DETAILED DESCRIPTION OF THE INVENTION

In accordance with the present invention a method is provided for the treatment of an animal, preferably a mammal, preferably a human with ophthalmologic disorders including glaucoma and reduced accommodation. Briefly the method of the present invention provides for a method of treatment of mammals with glaucoma or reduced accommodation that can be caused by age or certain age-related diseased states such as diabetes. The method provides for administration of classes of inhibitors of advanced glycation. The invention further provides for methods to monitor the improvement in the ocular condition during the course of the administration of compound.

Provided is a method of treating or ameliorating an indication of the invention in an animal, including a human, comprising administering an effective amount of (A) a compound of the formula I:
Y—Ar^{⊕}•X⁻ (I)
wherein:
a. Ar is a five or six membered heteroaryl ring having a first ring nitrogen and optionally second or third ring nitrogens, with the remaining ring atoms being carbon, oxygen, or sulfur, provided the first nitrogen of Ar is a quaternary nitrogen and Ar is not thiazolium, oxazolium or imidazolium;
b. Y is substituted on the first ring nitrogen, with the proviso that if Ar is pyrazole, indazole, (1,2,3)-triazole, benzotriazole, or (1,2,4)-triazole, the second ring nitrogen is substituted with
   1. alkyl or alkoxycarbonylalkylene;
   2. Ar* {wherein, consistent with the rules of aromaticity, Ar* is (and Ar², Ar³, Ar⁴ and Ar⁵ are) C₆ or C₁₀aryl or a 5- or 6-membered heteroaryl ring, wherein 6-membered heteroaryl ring contains one to three atoms of N, and the 5-membered heteroaryl ring contains from one to three atoms of N or one atom of O or S and zero to two atoms of N, each heteroaryl ring may be fused to a benzene, pyridine (which is omitted in some embodiments), pyrimidine, pyridazine, pyrazine, or (1,2,3)triazine (wherein the ring fusion is at a carbon-carbon double bond of Ar*) (in one embodiment, Ar* is (and Ar², Ar³, Ar⁴ and Ar⁵ are) C₆ or C₁₀aryl)}; or
   3. Ar*alkyl-, Ar*C(O)alkyl-, Ar*sulfonylalkyl-, or Ar*sulfinylalkyl-; and
c. Ar can be substituted on ring carbon atoms
   1. with one or more substituents independently selected from the group consisting ω-alkylenesulfonic acid, carbamoyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)— (in one embodiment, the substituents for Ar are (preferably exclusively) selected from the group consisting hydrogen, acylamino, alkanoyl, alkanoylalkyl, alkoxy, alkoxycarbonyl, alkoxycarbonylalkyl, alkyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid (—SO3H), alkylsulfonyl (alkylSO₂—), alkylsulfinyl (alkylSO—), alkylthio, trifluoromethyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—, wherein Ar* may be substituted by one or more substituents as set forth above); or
   2. two adjacent substitutions together with their ring carbons form a C₆- or C₁₀-aromatic fused ring system; or
   3. two adjacent substitutions together with their ring carbons form a C₅-C₇ fused cycloalkyl ring having up to two double bonds including the fused double bond of the Ar group (in one embodiment, no double bonds except the fused double bond), which cycloalkyl ring can be substituted by one or more of the group consisting of alkyl, alkoxycarbonyl, amino, aminocarbonyl, carboxy, fluoro, or oxo (in one embodiment, multiple substituents are located on different carbon atoms of the cycloalkyl ring, except in the case of alkyl, and fluoro substituents, which can be located on the same or different carbon atoms); or
   4. two adjacent substitutions together with their ring carbons form a fused five to eight membered heterocycle, wherein the ring fusion is at a carbon-carbon double bond of Ar, wherein the heterocycle consists of ring atoms selected from the group consisting of carbon, nitrogen, oxygen, and S(O)ₙ, wherein n=0, 1, or 2; or
   5. two adjacent substitutions together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur (in one embodiment, the substitution patterns are selected from options 1., 2. and 3.);
d. Y is:
   1. a group of the formula —CH(R⁵)-R⁶ (as preferred in one embodiment)
      (a) wherein R⁵ is hydrogen, alkyl-, cycloalkyl-, alkenyl-, alkynyl-, aminoalkyl-, hydroxy[C₁ to C₆]alkyl, dialkylaminoalkyl-, (N-[C₆ or C₁₀]aryl)(N-alkyl)aminoalkyl-, piperidin-1-ylalkyl-, pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperazin-1-ylalkyl, piperidin-1-ylalkyl, [C₆ or C₁₀]aryl, or independently the same as R⁶ (in one embodiment, hydrogen or alkyl);
      (b) wherein R⁶ is
         (1) hydrogen, alkyl (which in one embodiment may be substituted by alkoxycarbonyl)-, alkenyl, alkynyl, cyano-, cyanoalkyl-, or Rs, wherein Rs is a [C₆ or C₁₀]aryl or a heterocycle containing 4-10 ring atoms of which 1-3 are heteroatoms selected from the group consisting of oxygen, nitrogen and sulfur; or
         (2) a group of the formula —W—R⁷ [as preferred in one embodiment], wherein R⁷ is alkyl, alkoxy, hydroxy, or Rs [as preferred in one embodiment], wherein W is —C(═O)— or —S(O)₂—;
         (3) a group of the formula —W—OR⁸ wherein R⁸ is hydrogen or alkyl,
         (4) a group of the formula —CH(OH)Rs; or
         (5) a group of the formula —W—N(R⁹)R¹⁰, wherein
            (a) R⁹ is hydrogen and R¹⁰ is an alkyl or cycloalkyl, optionally substituted by
            (i) [C₆ or C₁₀]aryl, or
            (ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C1-C3)alkylenedioxy groups, or fused to a phenyl or pyridine ring, wherein the ring fusion is at a carbon-carbon double bond of the heteroaryl ring) (in one embodiment, which may or may not be in addition to the general substitutions, optionally substituted with one or more halo or (C₁-C₃)alkylenedioxy groups, or fused to a phenyl ring), or
            (iii) a heterocycle containing 4-10 ring atoms of which 1-3 are heteroatoms selected from the group consisting of oxygen, nitrogen and sulfur (in one embodiment, the R¹⁰ substituents are selected from (i) and (ii)); or
         (b) R⁹ is hydrogen or alkyl and R¹⁰ is Ar*; or
         (c) R⁹ is hydrogen or alkyl, R¹⁰ is a heterocycle containing 4-10 ring atoms of which 1-3 are heteroatoms are selected from the group consisting of oxygen, nitrogen and sulfur; or
         (d) R⁹ and R¹⁰ are both alkyl groups; or
         (e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆- or C₁₀)aryl, (C₆- or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy (in one embodiment,); or
         (f) R⁹ and R¹⁰ are both hydrogen (in one embodiment, R⁹ and R¹⁰ are selected from the (a), (b), (e) or (f) options); or
      2. —NH₂, and
e. X is a pharmaceutically acceptable anion, which may be absent if the compound provides a neutralizing salt,
(B) a pharmaceutically acceptable salt of the compound,
wherein aryl, Ar or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar C(O)NH—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, thiomorpholin-4-yl, piperidin-1-yl (the “Aryl General Substituents,” where the “Ar*” recited is Ar*, Ar², Ar³, Ar⁴ or Ar⁵, as appropriate) (in one embodiment, aryl or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar²C(O)—, Ar²C(O)NH—, Ar²O—, Ar²—, Ar²-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid (the “Aryl Preferred General Substitutions,” where the “Ar*” recited is Ar*, Ar², Ar³, Ar⁴ or Ar⁵, as appropriate)); and
wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*—, carboxy, dialkylamino, fluoro, fluoroalkyl, difluoroalkyl, hydroxy, mercapto, sulfamoyl, or trifluoromethyl (the “Heterocycle General Substituents,” where the “Ar*” recited is Ar*, Ar², Ar³, Ar⁴ or Ar⁵, as appropriate) (in one embodiment, heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylsulfonyl, alkylsulfinyl, alkylthio, Ar²C(O)—, Ar²O—, Ar²—, carboxy, fluoro, fluoroalkyl, difluoroalkyl, hydroxy, mercapto, sulfamoyl, or trifluoromethyl (the “Heterocycle Preferred General Substituents,” where the “Ar*” recited is Ar*, Ar², Ar³, Ar⁴ or Ar⁵, as appropriate)) (in one embodiment, multiple substituents are located on different atoms of the heterocyclic ring, with the proviso that alkyl, alkylcarbonyl, and fluoro substituents can be substituted on the same carbon atom of the heterocyclic ring).

In certain embodiments of the invention, if the compound of formula I has a core structure comprising a pyridinium ring having a 2-aryl-2-oxoethyl substitution at the 1 position, wherein the aryl can be substituted, and a formyl which may be substituted at the 3 position, one or both of the following applies: (1) the compound of formula VII differs from a salt of pyridinium compound having a 1-(2-aryl-2-oxoethyl), wherein the aryl can be substituted, and a formyl which may be substituted at the 3 position by at least one additional substitution at R¹⁴, R¹⁵ or R¹⁶, or (2) the aryl of 2-aryl-2-oxoethyl is phenyl and is substituted at the para position with an electron withdrawing group selected from fluoro, chloro, nitro, trifluoromethyl, and carbamoyl, and the compound used in a method of the invention is subject to the same restrictions. In certain embodiments, the compound used in a method of the invention.

The invention relates to compounds and pharmaceutical formulations including, without limitation, the compounds and formulations (compound and pharmaceutically acceptable excipient) thereof specifically recited below. In addition to the methods, compounds, and compositions thereof described herein, the invention provides methods or use in the treatments of the invention, or in the manufacture of a medicament for such therapeutic use.

Primary open angle glaucoma is characterized by an increase in intraocular pressure. The condition of open angle glaucoma is characterized by an increase in the pressure within a person's eye or eyes, called the intraocular pressure. The normal pressure is about 15 mmHg. Elevated pressures of 20-30 mm Hg create a strong risk of damage to the optic nerve and blindness.

Glucose reacts with proteins by a non-enzymatic, post-translational modification process called non-enzymatic glycosylation. The resulting sugar-derived adduct, the advanced glycosylation end product (AGE), matures to a molecular species that is reactive, and can readily bond to amino groups on adjacent proteins, resulting in the formation of AGE cross-links between proteins.

It has now been found that certain compounds that inhibit the formation of such sugar-derived adducts, or in some cases are believed to deactivate such adducts or break resulting crosslinks, can reduce intraocular pressure or ameliorate a trend towards elevated pressure.

Structural matrix proteins isolated from tissues of diabetics and aged individuals are more highly crosslinked than those from nondiabetics or younger individuals and are more resistant to both enzymatic and chemical hydrolysis in vitro. It is this cross-linked state of proteins that is believed to cause stiffness of tissues. The cleavage of AGE cross-links between proteins can provide a mechanism-based therapy for restoration of normal tissue function. An agent that cleaves AGE cross-links between proteins or inhibits their formation can restore more normal sieving function and movement to the trabecular meshwork.

In accordance with the present invention, methods for administering pharmaceutical compositions containing compounds have been developed for the treating glaucoma, intraocular pressure associated with glaucoma, and reduced accommodation. These agents are compounds of the general formula Y—Ar+X—(I), wherein Ar is a nitrogen containing, five or six-membered aromatic heterocycle as shown in the Summary section above.

Pharmaceutical compositions of the invention include administering an intraocular pressure decreasing amount of a compound of the formula I.

Compounds of the invention include compounds of the general formula Y—Ar⁺X—, wherein Ar is a nitrogen containing, five or six-membered aromatic heterocycle (heteroaryl). The nitrogen containing, five or six-membered aromatic heterocycle contains, consistent with the rules governing aromaticity, from 1 to 3 heteroatoms of N, O or S, with the proviso that Ar is not thiazole, oxazole, or imidazole.

The alkyl, and alkenyl groups referred to below include both C1 to C6 linear and branched alkyl and alkenyl groups, unless otherwise noted. Unless otherwise noted, alkoxy groups include linear or branched C1 to C6 alkoxy groups.

“Ar*” (consistent with the rules governing aromaticity) refers to a C₆ or C₁₀ aryl, or a 5 or 6 membered heteroaryl ring. The heteroaryl ring contains at least one and up to three atoms of N for the 6 membered heteroaryl ring. The 5 membered heteroaryl ring contains; (1) from one to three atoms of N, or (2) one atom of O or S and zero to two atoms of N. The aryl or heteroaryl is optionally substituted as set forth below. Nonlimiting examples of heteroaryl groups include: pyrrolyl, furanyl, thienyl, pyridyl, oxazolyl, pyrazolyl, pyrimidinyl, and pyridazinyl.

“Ar*” can be fused to either a benzene, pyridine, pyrimidine, pyridazine, or (1,2,3) triazine ring.

“Rs” refers to a C₆ or C₁₀ aryl group (wherein said aryl is optionally substituted as set forth below) or a heterocycle containing 4-10 ring members and 1-3 heteroatoms selected from the group consisting of oxygen, nitrogen and sulfur (wherein said heterocycle is optionally substituted as set forth below).

As used herein, C₆ or C₁₀ aryl groups and heterocycle containing 4 to 10 ring members are monocyclic or bicyclic.

In certain embodiments of the invention, Ar contains adjacent substitutions on ring carbons that together with their ring carbons (the carbons to which the adjacent substitution) form a fused C5 to C7 cycloalkyl ring having up to two double bonds including the fused double bond. The cycloalkyl ring can be substituted by one or more of the group consisting of alkyl, alkoxycarbonyl, amino, aminocarbonyl, carboxy, fluoro, and oxo substituents. One of ordinary skill in the art will recognized that where cycloalkyl groups contain double bonds, the sp² hybridized carbon atoms can contain only one substituent (which can not be amino- or oxo-). Sp³ hybridized carbon atoms in the cycloalkyl ring can be geminally substituted with the exception that (1) two amino groups and (2) one amino and one fluoro group can not be substituted on the same sp³ hybridized carbon atom.

In certain embodiments of the invention, Ar contains adjacent substitutions on ring carbons that together with their ring form a five to eight membered heterocycle (i.e. a bicyclic heterocycle is formed). In these embodiments the heterocycle formed by the adjacent substituents is preferably not aromatic. (Alternative embodiments refer to an aromatic heterocyclic ring, referred to as heteroaryl, formed by adjacent substitutions of Ar.) Particular compounds within embodiments containing a heterocyclic ring fused to Ar contain sulfur atoms in the fused ring. These sulfur atoms in these particular compounds can exist in various oxidation states, as S(O)ₙ, where n is 0, 1, or 2.

In certain embodiments of the invention, Ar contains a Y group which can be —CH(R⁵)-R⁶. In those embodiments wherein R⁵ is alkenyl, preferably alkenyl is —C═C—R^{G}, where R^{G} is alkyl, H, or hydroxy(C₁-C₆)alkyl. In those embodiments wherein R⁵ is alkynyl, preferably alkynyl is —C≡C—R^{H}, wherein R^{H} is alkyl, hydrogen, or hydroxy(C₁-C₆)alkyl.

Aryl, Ar, or Ar* can be substituted with, in addition to any substitutions specifically noted, one or more substituents selected from the group consisting of acylamino, acyloxyalkyl, alkanoyl, alkanoylalkyl, alkenyl, alkoxy, alkoxycarbonyl, alkoxycarbonylalkyl, alkyl, alkylamino, (C1-C3)alkylenedioxy, alkylsulfonyl [alkylS(O)₂—], alkylsulfinyl [alkylS(O)—], ω-alkylenesulfonic acid [-alkylSO₃H], alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C2-C6)hydroxyalkyl, mercapto, nitro, sulfamoyl, sulfonic acid (—SO₃H), 1-pyrrolidinyl-, 4-[C6 or C10]arylpiperazin-1-yl-, 4-aryl[C6 or C10]piperidin-1-yl, azetidin-1-yl, morpholin-4-yl, thiomorpholin-4-yl, piperazin-1-yl, and piperidin-1-yl.

Heterocycles, except those of Ar and Ar* can be substituted with, in addition to any substitutions specifically noted, acylamino, alkanoyl, alkoxy, alkoxycarbonyl, alkoxycarbonylalkyl, alkyl, alkylamino, alkylsulfonyl, alkylsulfinyl, alkylthio, amino, Ar*C(O)—, Ar*O—, Ar*—, carboxy, dialkylamino, fluoro, fluoroalkyl, difluoroalkyl, hydroxy, mercapto, sulfamoyl, or trifluoromethyl. Preferably, multiple substituents are located on different atoms of the heterocyclic ring, with the proviso that alkyl, alkoxycarbonyl, and fluoro substituents can be substituted on the same carbon atom of the heterocyclic ring. Heterocycles can be substituted with one or more substituents.

The halo atoms can be fluoro, chloro, bromo or iodo. Chloro and fluoro are preferred substituents for aryl substitutions.

For the purposes of this invention, the compounds of formula (I) are formed as biologically and pharmaceutically acceptable salts. Useful salt forms include the halides (particularly bromides and chlorides), tosylates, methanesulfonates, brosylates, fumarates, maleates, succinates, acetates, mesitylenesulfonates, and the like. Other related salts can be formed using similarly non-toxic, and biologically and pharmaceutically acceptable anions.

In certain embodiments of the invention, X (a pharmaceutically acceptable anion) can be absent when the molecule provides anionic moieties such as carboxylates and sulfonates. In these embodiments the compounds exist as zwitterions.

Salt formation of the nitrogen containing aromatic heterocycle (Ar) is achieved by either by alkylation or by amination (—NH₂) of a ring nitrogen atom.

Compounds of the general formula Y—Ar+^{·}X—, can be prepared either by chemical syntheses well known in the art or by the methods described below. In addition, certain of the aromatic heterocycles, useful as intermediates for the preparation of compounds of the invention, are well-known and readily available from chemical supply houses or can be prepared by synthetic schemes specifically published therefor. The chemical reagents shown in the schemes below provide nonlimiting examples of means well known the art to carry out the reaction steps shown below.

Preferred five-membered ring heterocycles of the invention include positively charged pyrazoles, triazoles (both 1,2,3 and 1,2,4-triazoles), oxadiazoles (1,2,4), and thiadiazoles (both 1,2,3 and 1,3,4) that are alkylated at a ring N atom. Preferred compounds of the invention also include the corresponding benzo-fused analogs of the N-alkylated five-membered ring heterocycles. For example, preferred compounds of the invention includes N-alkylated indazoles, benzotriazoles and benzothiadiazoles (1,2,3).

The invention does not include positively charged analogs of the five-membered nitrogen containing heteroaromatics thiazole, oxazole, and imidazole (i.e. thiazoliums, imidazoliums, and oxazoliums).

Preferred six-membered ring heterocycles include positively charged, ring N-alkylated pyridazines, pyridines, and pyrimidines. In addition, preferred compounds of the invention include the corresponding benzo-fused analogs of the N-alkylated six-membered ring heterocycles. For example quinolines, isoquinolines, quinazolines, cinnolines, and phthalazines alkylated at a ring N atoms are preferred compounds of the invention.

In one embodiment Ar can substituted on ring carbon atoms:
1. with one or more substituents independently selected from the group consisting hydrogen, acylamino, alkanoyl, alkanoylalkyl, alkoxy, alkoxycarbonyl, alkoxycarbonylalkyl, alkyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid (—SO₃H), alkylsulfonyl (alkylSO₂—), alkylsulfinyl (alkylSO—), alkylthio, trifluoromethyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—, wherein Ar* may be substituted by one or more substituents as set forth above; or
2. two adjacent substitutions together with their ring carbons form a C₆- or C₁₀-aromatic fused ring system; or
3. two adjacent substitutions together with their ring carbons form a C₅-C₇ fused cycloalkyl ring having no double bonds except the fused double bond of the Ar group, which cycloalkyl ring can be substituted by one or more of the group consisting of alkyl, amino, aminocarbonyl, carboxy, fluoro, or oxo, wherein multiple substituents are located on different carbon atoms of the cycloalkyl ring, except in the case of alkyl, and fluoro substituents, which can be located on the same or different carbon atoms [in one embodiment, the substitutions do not include amino].

In another embodiment, Y is:
1. a group of the formula —CH(R⁵)—R⁶
   (a) R⁵ is hydrogen or alkyl;
   (b) wherein R⁶ is
      (1) hydrogen, alkyl , alkenyl, alkynyl, cyano, cyanoalkyl, or Rs; or
      (2) a group of the formula —W—R⁷, wherein R⁷ is alkyl, alkoxy, hydroxy, or Rs, wherein W is —C(═O)— or —S(O)₂—;
      (3) a group of the formula —W—OR⁸ wherein R⁸ is hydrogen or alkyl,
      (4) a group of the formula —CH(OH)Rs; or
      (5) a group of the formula —W—N(R⁹)R¹⁰, wherein
         (a) R⁹ is hydrogen and R¹⁰ is an alkyl or cycloalkyl, optionally substituted by
         (i) [C₆ or C₁₀]aryl, or
         (ii) a 5- or 6-membered heteroaryl ring that can, in addition to the general substitutions, be optionally substituted with one or more halo or (C₁-C₃)alkylenedioxy groups, or fused to a phenyl ring, or
         (b) R⁹ is hydrogen or alkyl and R¹⁰ is Ar*; or
         (e) R⁹ and R¹⁰ together with N form a heterocycle wherein any heteroaryl substitution thereto can be optionally substituted, in addition to the general substitutions, with one or more halo or (C₁-C₃)alkylenedioxy; or
         (f) R⁹ and R¹⁰ are both hydrogen.

In still another embodiment, Y is NH₂—.

In another embodiment, the substitutions selected from the listing above reading “wherein aryl, AR or Ar* can be substituted . . . ” do not include alkylamino, amino, dialkylamino, 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, thiomorpholin-4-yl, or piperidin-1-yl.

In still another embodiment, the substitutions selected from the listing above reading “wherein heterocycles, except those of Ar or Ar* . . . ” do not include alkylamino, amino or dialkylamino. Preferably, multiple substituents are located on different atoms of the heterocyclic ring, with the proviso that alkyl, alkylcarbonyl, and fluoro substituents can be substituted on the same carbon atom of the heterocyclic ring.

In another embodiment Y—Ar^{⊕}•X⁻ is 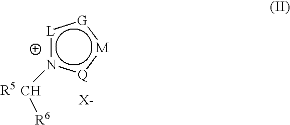
wherein G, L, M, and Q are independently O, S, N, N—R^{a}, C, C—R^{b}, C—R^{c}, C—R^{d}, wherein no more than one of G, L, M, or Q is O or S;
wherein
1. R⁵ is H;
2. R⁶ is
   (1) cyano or
   (2) a group of the formula —W—R⁷, wherein R is alkyl or Rs, and W is —C(═O)— or —S(═O)—;
   (3) a group of the formula —W—N(R⁹)R¹⁰, wherein
      (a) R⁹ is hydrogen and R¹⁰ is an alkyl or cycloalkyl, optionally substituted by
         (i) [C₆ or C₁₀]aryl, or
         (ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring);
3. R^{a} is alkyl, Ar*, Ar*alkyl, alkoxycarbonylalkylene-, Ar*C(O)alkyl-, Ar*sulfonylalkyl-, or Ar*sulfinylalkyl-; and
4. R^{b}, R^{c}, and R^{d} are
   (a) independently selected from the group consisting hydrogen, acylamino, acyloxyalkyl, alkanoyl, alkanoylalkyl, alkenyl, alkoxy, alkoxycarbonyl, alkoxycarbonylalkyl, alkyl, alkylamino, (C1-C3)alkylenedioxy, alkylsulfonyl, alkylsulfinyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO₃H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, piperidin-1-yl;
   (b) wherein any two of R^{b}, R^{c}, and R^{d} are adjacent, together with their ring carbons form a C₆ or C₁₀ aromatic fused ring system;
   (c) wherein any two of R^{b}, R^{c}, and R^{d} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;
   (d) wherein any two of R^{b}, R^{c}, and R^{d} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0, 1, or 2; and
   (e) wherein any two of R^{b}, R^{c}, and R^{d} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur.
      In one embodiment of the invention defined by formula II, Ar is not tetrazole or pyrrole. In one embodiment, aryl, Ar or Ar* is substituted with, in addition to any substitutions specifically noted above, one or more substituents selected from the group consisting of hydrogen, alkyl, amino, dialkylamino, 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, piperidin-1-yl. The compound of formula II can be further defined in preferred embodiments as pursuant to one of the following:

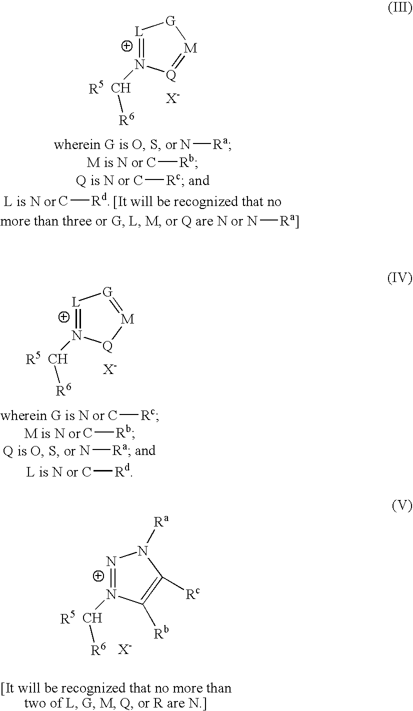

In another embodiment, Y—Ar^{⊕}•X⁻ is 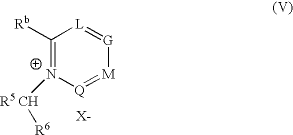
wherein L, G, M, Q, or R are independently N, C—R^{c}, C—R^{d}, C—R^{e}, C—R^{f};
wherein
1. R⁵ is H;
2. R⁶ is
   (1) cyano or
   (2) a group of the formula —W—R⁷, wherein R⁷ is alkyl or Rs, and W is —C(═O)— or —S(═O)—;
   3. R^{b}, R^{c}, R^{d}, and R^{e} are
      (a) independently selected from the group consisting hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid (SO3H), 1-pyrrolidinyl-, 4-[C₆ or C₁₀]arylpiperazin-1-yl-, 4-[C₆ or C₁₀]arylpiperidin-1-yl, azetidin-1-yl, and morpholin-4-yl, piperidin-1-yl;
      (b) where any two of R^{b}, R^{c}, R^{d}, and R^{e} are adjacent, together with their ring carbons form a C₆- or C₁₀-aromatic fused ring system;
      (c) where any two of R^{b}, R^{c}, R^{d}, and R^{e} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;
      (d) wherein any two of R^{b}, R^{c}, R^{d}, and R^{e} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0, 1, or 2;
      (e) wherein any two of R^{b}, R^{c}, R^{d} and R^{e} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur, and
         wherein Ar has no more than three nitrogen atoms in the ring. In one embodiment, Ar is substituted with amino, or two amino groups.

In one embodiment, any cylcloalkyl from any two adjacent substitutions to Ar that together with their ring carbons that form a C₅-C₇ fused cycloalkyl is not substituted with amino. In one embodiment, R⁵ is hydrogen, alkyl-, cycloalkyl-, alkenyl-, alkynyl-, hydroxy[C₁ to C₆]alkyl, [C₆ or C₁₀]aryl, or independently the same as R⁶. In one embodiment, any 5- or 6-membered heteroaryl ring substituted on R¹⁰ is not substituted with 1-pyrrolidinyl, 4-[C₆ or C¹⁰]arylpiperazin-1-yl, 4-[C₆ or C₁₀]arylpiperidin-1-yl, azetidin-1-yl, morpholin-4-yl, thiomorpholin-4-yl or piperidin-1-yl, and is not fused or pyridine ring. In another embodiment, any heterocycle formed from R⁹ and R¹⁰ is not substituted with 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl or piperidin-1-yl.

The invention provides a compound of formula VI: 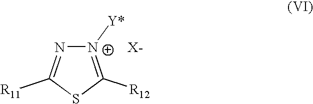
wherein
a. one of R¹¹ and R¹² is hydrogen [preferably R¹² is hydrogen] and the other is selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar², Ar²-alkyl, Ar²—O, Ar²SO₂—, Ar²SO—, Ar²S—, Ar²SO₂NH—, Ar²NH, (N—Ar²)(N-alkyl)N—, Ar²C(O)—, Ar²C(O)NH—, Ar²NH—C(O)—, or (N—Ar²)(N-alkyl)N—C(O)— [in one embodiment, independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfinyl, alkylthio, trifluoromethyl, Ar², Ar²-alkyl, Ar²—O, Ar²SO₂—, Ar²SO—, Ar²S—, Ar²SO₂NH—, Ar²NH, (N—Ar²)(N-alkyl)N—, Ar²C(O)—, Ar²C(O)NH—, Ar²NH—C(O)—, and (N—Ar²)(N-alkyl)N—C(O)—];
b. Y* is a group of the formula —CH(R⁵)-R⁶ wherein
   (a) R⁵ is hydrogen, alkyl- , cycloalkyl-, alkenyl-, alkynyl-, aminoalkyl-, dialkylaminoalkyl-, (N—[C₆ or C₁₀]aryl)(N-alkyl)aminoalkyl-, piperidin-1-ylalkyl-, 1-pyrrolidinylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶ [in one embodiment, R⁵ is hydrogen or alkyl];
   (b) R⁶ is
      (1) cyano or Rs, wherein Rs is a [C₆ or C₁₀]aryl or a heterocycle containing 4-10 ring atoms of which 1-3 are heteroatoms selected from the group consisting of oxygen, nitrogen and sulfur;
   (2) a group of the formula —W-Rs, wherein W is —C(═O)— or —S(O)ₙ— where n=1 or 2;
   (3) a group of the formula —W—N(R⁹)R¹⁰, wherein
      [a] R⁹ is hydrogen and R¹⁰ is an alkyl or cycloalkyl, optionally substituted by
         (i) [C₆ or C₁₀]aryl, or
         (ii) a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl, halo or (C₁-C₃)alkylenedioxy groups, or fused to a substituted phenyl or pyridine ring, wherein the ring fusion is at a carbon-carbon double bond of the heteroaryl ring [in one embodiment, such heteroaryl ring can be optionally substituted with one or more halo or (C₁-C₃)alkylenedioxy groups, or fused to a substituted phenyl], or
         (iii) a heterocycle containing 4-10 ring atoms of which 1-3 are heteroatoms selected from the group consisting of oxygen, nitrogen and sulfur; or
      [b] R⁹ is hydrogen or lower alkyl and R¹⁰ is Ar²; or
      [c] R⁹ is hydrogen or lower alkyl, and R¹⁰ is a heterocycle containing 4-10 ring atoms of which 1-3 are heteroatoms are selected from the group consisting of oxygen, nitrogen and sulfur, said heterocycle; or
      [d] R⁹ and R¹⁰ are both alkyl groups; or
      [e] R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy [in one embodiment, such heteroaryl can be optionally substituted, in addition to the general substitutions, with one or more halo or (C₁-C₃)alkylenedioxy]; or
      [f] R⁹ and R¹⁰ are both hydrogen; and
c. X is a pharmaceutically acceptable anion, or
(B) a pharmaceutically acceptable salt of the compound,
wherein aryl or Ar² can be substituted with, in addition to any substitutions specifically noted, one or more substituents selected from the Aryl General Substitutions [in one embodiment, one or more substituents selected from the Aryl Preferred General Substituions];
wherein heterocycles, except those of Ar², can be substituted with, in addition to any substitutions specifically noted, the Heterocyle General Substitutions [in one embodiment, the Heterocycle Preferred General Substituents];
wherein the compound of formula VI differs from a salt of 3-[2-(4-bromophenyl)-2-oxoethyl]-1,3,4-thiadiazolium by one or more of the lack or replacement of the 4-bromo substitution, or the presence of one or more additional substitutions [preferably the differences in substitutions total two or more]; and
wherein the compound of formula VI differs from a salt of 3 -(phenylmethyl)-1,3,4-thiadiazolium by the presence of one or more additional substitutions [preferably the differences in substitutions total two or more].

3-[2-(4-Bromophenyl)-2-oxoethyl]-1,3,4-thiadiazolium bromide and 3-(phenylmethyl)-1,3,4-thiadiazolium chloride are described in Haug et al., Liebigs Ann. Chem. 1988(6): 605-7, as intermediates for forming spirocyclic compounds. 3-(Phenylmethyl)-1,3,4-thiadiazolium chloride is also mentioned in JP 04081597 (issued 24 Dec. 1992), it is believed as a reagent involved in converting formaldehyde to dendroketose, and in Takamizawa et al., Chem. Pharm. Bull. 18(6):1201-10, 1970, an article on the reaction of 1,3,4-thiadiazolium halides with dialkyl acylphosphonates in the presence of Et₃N to give 1,3,4-thiadiazine derivatives, accompanied by ring expansion.

The invention further provides a compound of formula VII: 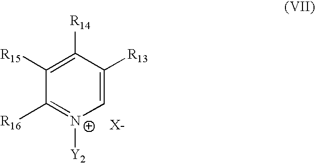
wherein
a. R¹³, R⁴, R¹⁵ and R¹⁶
   1. are independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar³, Ar³-alkyl, Ar³—O, Ar³SO₂—, Ar³SO—, Ar³S—, Ar³SO₂NH—, Ar³NH, (N—Ar³)(N-alkyl)N—, Ar³C(O)—, Ar³C(O)NH—, Ar³NH—C(O)—, and (N—Ar³)(N-alkyl)N—C(O)—, or together R₁ and R₂ comprise methylenedioxy [in one embodiment, independently selected from hydrogen, acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, allyl, ω-alkylenesulfonic acid, carbamoyl, carboxy, carboxyalkyl, cycloalkyl, halo, hydroxy, (C₂-C₆)hydroxyalkyl, mercapto, nitro, sulfamoyl, sulfonic acid, alkylsulfonyl, alkylsulfinyl, alkylthio, trifluoromethyl, Ar³, Ar³-alkyl, Ar³—O, Ar³SO₂—, Ar³SO—, Ar³S—, Ar³SO₂NH—, Ar³NH, (N—Ar³)(N-alkyl)N—, Ar³C(O)—, Ar³C(O)NH—, Ar³NH—C(O)—, and (N—Ar³)(N-alkyl)N—C(O)—]; or
   2. form, with an adjacent pair from R¹³, R¹⁴, R¹⁵ and R¹⁶ together with their ring carbons, a C₆- or C₁₀- aromatic fused ring system; or
   3. form, with an adjacent pair from R¹³, R¹⁴, R¹⁵ and R¹⁶, together with their ring carbons, a C₅-C₇ fused cycloalkyl ring having up to two double bonds including the fused double bond of the pyridinium containing ring, which cycloalkyl ring can be substituted by one or more of the group consisting of alkyl, alkoxycarbonyl, amino, aminocarbonyl, carboxy, fluoro, or oxo substituents [in one embodiment, the substitutions do not include amino]; or
   4. form, with an adjacent pair from R¹³, R¹⁴, R¹⁵ and R¹⁶, together with their ring carbons, a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring may be optionally substituted with one or more 1-pyrrolidinyl-, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups [in one embodiment, the optional substitutions are one or more halo or (C₁-C₃)alkylenedioxy groups]; or
   5. form, with an adjacent pair from R¹³, R¹⁴, R¹⁵ and R¹⁶, together with their ring carbons, a five to eight membered heterocycle, wherein the heterocycle consists of ring atoms selected from the group consisting of carbon, nitrogen, and S(O)ₙ, where n=0, 1, or 2;
b. Y² is a group of the formula —CH(R⁵)-R⁶ wherein
   (a) R⁵ is hydrogen, alkyl-, cycloalkyl-, alkenyl-, alkynyl-, aminoalkyl-, dialkylaminoalkyl-, (N-[C₆ or C₁₀]aryl)(N-alkyl)aminoalkyl-, piperidin-1-ylalkyl-, 4-pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀] arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶ [in one embodiment, R⁵ is hydrogen or alkyl];
   (b) R⁶ is
      (1) cyano or Rs, wherein W is —C(═O)— or —S(O)ₙ— where n=1 or 2, and Rs is a C₆ or C₁₀ aryl or a heterocycle containing 4-10 ring atoms of which 1-3 are heteroatoms selected from the group consisting of oxygen, nitrogen and sulfur;
      (2) a group of the formula —W-Rs, wherein W is —C(═O)— or —S(O)ₙ— where n=1 or2;
      (3) a group of the formula —W—N(R⁹)R¹⁰, wherein
         [a] R⁹ is hydrogen and R¹⁰ is an alkyl or cycloalkyl, optionally substituted by
            (i) [C₆ or C₁₀]aryl, or
            (ii) a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring [in one embodiment, the optional substitutions are one or more halo or (C₁-C₃)alkylenedioxy groups, or fused to a substituted phenyl], or
            (iii) a heterocycle containing 4-10 ring atoms of which 1-3 are heteroatoms selected from the group consisting of oxygen, nitrogen and sulfur; or
         [b] R⁹ is hydrogen or lower alkyl and R¹⁰ is Ar³; or
         [c] R⁹ is hydrogen or lower alkyl, and R¹⁰ is a heterocycle containing 4-10 ring atoms of which 1-3 are heteroatoms are selected from the group consisting of oxygen, nitrogen and sulfur, said heterocycle; or
         [d] R⁹ and R¹⁰ are both alkyl groups; or
         [e] R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each such heteroaryl can be optionally substituted, in addition to the general substitutions, with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy [in one embodiment, the optional substituents to the heteroaryl are one or more halo or (C₁-C₃)alkylenedioxy]; or
         [f] R⁹ and R¹⁰ are both hydrogen;
c. X is a pharmaceutically acceptable anion, or
(B) a pharmaceutically acceptable salt of the compound,
wherein aryl or Ar³ can be substituted with, in addition to any substitutions specifically noted, one or more substituents selected from the Aryl General Substituents or the Aryl Preferred General Substituents;
wherein heterocycles, except those of Ar, can be substituted with, in addition to any substitutions specifically noted, the Heterocycle General Substituents or the Heterocycle Preferred General Substituents; and
wherein, if the compound of formula VII has a core structure comprising a pyridinium ring having a 2-aryl-2-oxoethyl substitution at the 1 position, wherein the aryl can be substituted, and a formyl which may be substituted at the 3 position, one or both of the following applies:
   the compound of formula VII differs from a salt of pyridinium compound having a 1-(2-aryl-2-oxoethyl), wherein the aryl can be substituted, and a formyl which may be substituted at the 3 position by at least one additional substitution at R¹⁴, R¹⁵ or R¹⁶, or
   the aryl of 2-aryl-2-oxoethyl is phenyl and is substituted at the para position with an electron withdrawing group selected from fluoro, chloro, nitro, trifluoromethyl, , and carbamoyl; and
wherein the compound of formula VII differs from a salt of 1-[2-(4-methylphenyl)-2-oxoethyl]-pyridinium by one or more of the lack or replacement of the methyl substitution, or the presence of one or more additional substitutions [preferably the differences in substitutions total two or more].

Sankaranarayanan, WO 01/25209 describes certain pyridinium compounds substituted on the 1 (N) position 2-aryl-2-oxoethyl substitutions and derivative of formyl at the 3 position. 1-[2-(4-methylphenyl)-2-oxoethyl]-pyridinium chloride is described in J. Med. Chem. 32: 2301-6, 1989, as an inactive member of a series of compounds that sought to explore the glucose lowering effect of, particularly, certain imidazolium compounds.

The invention further provides a compound of formula VIII: 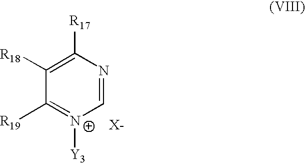
wherein
a. R¹⁷ R¹⁸ and R¹⁹
   1. are independently selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁴, Ar⁴-alkyl, Ar⁴—O, Ar⁴SO₂—, Ar⁴SO—, Ar⁴S—, Ar⁴SO₂NH—, Ar⁴NH, (N—Ar⁴)(N-alkyl)N—, Ar⁴C(O)—, Ar⁴C(O)NH—, Ar⁴NH—C(O)—, and (N—Ar⁴)(N-alkyl)N—C(O)—, or together R₁ and R₂ comprise methylenedioxy; or
   2. form, with an adjacent pair from R¹⁷, R¹⁸ and R¹⁹, together with their ring carbons, a C₆- or C₁₀- aromatic fused ring system; or
   3. form, with an adjacent pair from R¹⁷, R¹⁸ and R¹⁹, together with their ring carbons, a C₅-C₇ fused cycloalkyl ring having up to two double bonds including the fused double bond of the pyridinium containing ring, which cycloalkyl ring can be substituted by one or more of the group consisting of alkyl, alkoxycarbonyl, amino, aminocarbonyl, carboxy, fluoro, or oxo substituents [in one embodiment, the substitutions do not include amino; or
   4. form, with an adjacent pair from R¹⁷, R¹⁸ and R¹⁹, together with their ring carbons, a 5- or 6-membered heteroaryl ring, wherein the 6-membered heteroaryl ring contains one to three atoms of N, and the 5-membered heteroaryl ring contains from one to three atoms of N or one atom of O or S and zero to two atoms of N, each heteroaryl ring may be optionally substituted with one or more 1-pyrrolidinyl-, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups [in one embodiment, the optional substitutions are one or more halo or (C₁-C₃)alkylenedioxy groups]; or
   5. form, with an adjacent pair from R¹⁷, R¹⁸ and R¹⁹, together with their ring carbons, a five to eight membered heterocycle, wherein the heterocycle consists of ring atoms selected from the group consisting of carbon, nitrogen, and S(O)ₙ, where n=0, 1, or 2;
b. Y³ is a group of the formula —CH(R⁵)-R⁶ wherein
   (a) R⁵ is hydrogen, alkyl-, cycloalkyl-, alkenyl-, alkynyl-, aminoalkyl-, dialkylaminoalkyl-, (N-[C₆ or C₁₀]aryl)(N-alkyl)aminoalkyl-, piperidin-1-ylalkyl-, 4-pyrrolidin-1-ylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶ [in one embodiment, R⁵ is hydrogen or alkyl];
   (b) R⁶ is phenyl substituted on the para position with chloro or fluoro;
c. X is a pharmaceutically acceptable anion, or
(B) a pharmaceutically acceptable salt of the compound,
wherein aryl (including phenyl) or Ar⁴ can be substituted with, in addition to any substitutions specifically noted, one or more general substituents selected from the Aryl General Substitutions or Aryl Preferred General Substitutions; and
wherein heterocycles, except those of Ar⁴, can be substituted with, in addition to any substitutions specifically noted, the Heterocycle General Substitutions or Heterocycle Preferred General Substitutions;
wherein, in one embodiment, if Y has a core structure of phenyl substituted at the para position with chloro, then the compound of formula VIII differs from a salt of 1-[2-(4-bromophenyl)-2-oxoethyl]-5-cyano-pyrimidinium by a substitution difference of more than the cyano (which is not within the scope of R¹⁸).

1-[2-(4-Chlorophenyl)-2-oxoethylide]-5-cyano-pyrimidinium, 2-(4-Nitrophenyl)-2-oxoethylide)-pyrimidinium and 2-(4-Nitrophenyl)-2-oxoethyl)-pyrimidinium bromide are described in U.S. Pat. Nos. 3,836,352 and 3,702,361 as herbicides. 1-[2-(4-Bromophenyl)-2-oxoethyl]-4-(4-methylphenyl)-pyrimidinium bromide is is available from the Sigma-Aldrich Rare Chemical Library. 1-[2-[3,4-bis(acetyloxy)phenyl]-2-oxoethyl]-4-methylthio-pyrimidinium chloride is described in EP 304155 and corresponding U.S. Pat. No. 5,013,730, as in intermediate for making cephalosporin compounds. 4-(3-Methylphenyl)-1-[2-(2-nitrophenyl)-2-oxoethyl]-pyrimidinium bromide has a registry number of 373638-66-5.

The invention further provides a compound of formula IX: 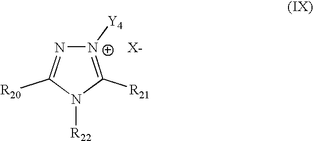
wherein
a. one of R²⁰ and R²¹ is hydrogen, and the other is selected from hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, allyl, amino, ω-alkylenesulfonic acid, carbamoyl, carboxy, carboxyalkyl, cycloalkyl, dialkylamino, halo, hydroxy, (C₂-C₆)hydroxyalkyl, mercapto, nitro, sulfamoyl, sulfonic acid, alkylsulfonyl, alkylsulfinyl, alkylthio, trifluoromethyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁵, Ar⁵-alkyl, Ar⁵—O, Ar⁵SO₂—, Ar⁵SO—, Ar⁵S—, Ar⁵SO₂NH—, Ar⁵NH, (N—Ar⁵)(N-alkyl)N—, Ar⁵C(O)—, Ar⁵C(O)NH—, Ar⁵NH—C(O)—, or (N—Ar⁵)(N-alkyl)N—C(O)—;
b. R²² is acylamino, acyloxyalkyl, alkanoylalkyl, alkenyl, alkoxycarbonyl, alkoxycarbonylalkyl, alkyl, allyl, carbamoyl, carboxyalkyl, dialkylamino, (C₂-C₆)hydroxyalkyl, azetidin-1-yl, morpholin-4-yl, thiomorpholin-4-yl, piperidin-1-yl, 4-[C₆ or C₁₀]arylpiperidin-1-yl, 4-[C₆ or C₁₀]arylpiperazin-1-yl, Ar⁵, Ar⁵ alkyl, Ar⁵—O, Ar⁵SO₂—, Ar⁵SO—, Ar⁵S—, Ar⁵SO₂NH—, Ar⁵NH, (N—Ar⁵)(N-alkyl)N—, Ar⁵C(O)—, Ar⁵C(O)NH—, Ar⁵NH—C(O)—, or (N—Ar⁵)(N-alkyl)N—C(O)—;
c. Y⁴ is a group of the formula —CH(R⁵)-R⁶ wherein
   (a) R⁵ is hydrogen, alkyl-, cycloalkyl-, alkenyl-, alkynyl-, aminoalkyl-, dialkylaminoalkyl-, (N-[C₆ or C₁₀]aryl)(N-alkyl)aminoalkyl-, piperidin-1-ylalkyl-, 1-pyrrolidinylalkyl, azetidinylalkyl, 4-alkylpiperazin-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperidin-1-ylalkyl, [C₆ or C₁₀]aryl, or independently the same as R⁶;
   (b) R⁶ is
      (1) cyano;
      (2) a group of the formula —W-Rs, wherein W is —C(═O)— or —S(O)ₙ— where n=1 or 2, and wherein Rs is a [C₆ or C₁₀]aryl or a heterocycle containing 4-10 ring atoms of which 1-3 are heteroatoms selected from the group consisting of oxygen, nitrogen and sulfur; and
d. X is a pharmaceutically acceptable anion, or
(B) a pharmaceutically acceptable salt of the compound,
wherein aryl (including phenyl) or Ar⁵ can be substituted with, in addition to any substitutions specifically noted, one or more general substituents selected from the Aryl General Substitutions or Aryl Preferred General Substitutions; and
wherein heterocycles, except those of Ar⁵, can be substituted with, in addition to any substitutions specifically noted, the Heterocycle General Substitutions or Heterocycle Preferred General Substitutions;

The invention further relates to the pharmaceutical compositions of the compounds specifically recited comprising such compounds with a pharmaceutically acceptable excipient, and effecting the methods of the invention with these compounds.

In general, compounds of the general formula Y—Ar+^{·}X—, wherein Y is as described above; and Ar is a nitrogen containing five or six-membered aromatic heterocycle, can be prepared by alkylation of the heterocycle under suitable alkylating conditions. By way of example, a 3-methyl (1,2,3)thiadiazolium salts can be prepared by N-alkylation of (1,2,3)thiadiazole with suitable alkylating agents, such as methyl iodide or methyl p-toluenesulfonic acid ester (Wolff, Kopitzsch Justus Liebigs Ann. Chem., 1904, 333, 20 and Adachi, J.; Takahat, H.; Nomura, K; Masuda, K. Chem. Pharm. Bull., 1983, 31(5) 1746-1750). In another example of the invention, 1-methyl triazole can be alkylated with benzyl iodide to give a compound wherein R⁵ is hydrogen and R⁶ is phenyl and Ar is a 3-methyl-(1,2,3)triazole (i.e. 1-benzyl-3-methyltriazolium iodide) (J. Am. Chem Soc., 1955, 77, 1703).

Generally, the compounds of the invention are synthesized by reacting Ar with Y—X, where X is a leaving group.

As is known in the art, alkylation of heterocycles containing more than one nitrogen atom in the ring (e.g., pyridazine, pyrazole, thiadiazole) can often lead to isomeric mixtures of N-alkylated products. In these cases, the isomers can be separated by any separation known in the art including fractional recrystallization, column chromatography, and the like. By way of example, alkylation of 4-substituted pyridazines can lead to mixture of pyridazines as shown below in Scheme 1. The isomeric N-alkylated pyridazine can be separated by the above mentioned techniques to provide compounds of the invention.

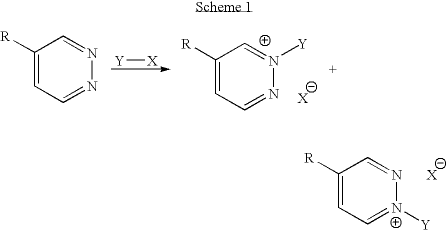

Those of ordinary skill in the art will recognize that some 5-membered aromatic heterocycles require substitution of two different nitrogen atoms in the heterocycle to effect quaternization. Five-membered ring heterocycles of this type include pyrazoles, (1,2,4)-triazoles, and (1,2,3)-triazoles (and benzofused analogs such as indazole and benzotriazole). The incorporation of one alkyl group in the heterocycle can be accomplished either by the use of a suitable N-alkylated acyclic precursor for ring formation, or by alkylation of the intact heterocycle. For instance, an alkyl pyrazole intermediate can be prepared by condensation of an alkyl hydrazine and a 1,3-dicarbonyl compound, or by alkylation with a pyrazole using suitable alkylating conditions (Scheme 2).

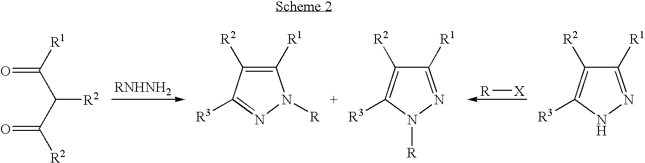

In general, compounds of the general formula Y—Ar+^{·}X-, wherein Y is CH(R⁵)—C(O)—R⁷ (wherein R⁵ and R⁷ is as described above; G, L, M, and Q are independently C, N, S or O; and X is a halide) can be prepared according to the synthetic route depicted in Scheme 3. An acetyl derivative with a suitable α leaving group, for example, an α-halo acetyl derivative, can be used to N-alkylate a suitably substituted aromatic heterocycle. The alkylation reaction can be conducted at elevated temperatures in a suitable solvent, for example, acetonitrile, acetone, or ethanol, or without solvent.

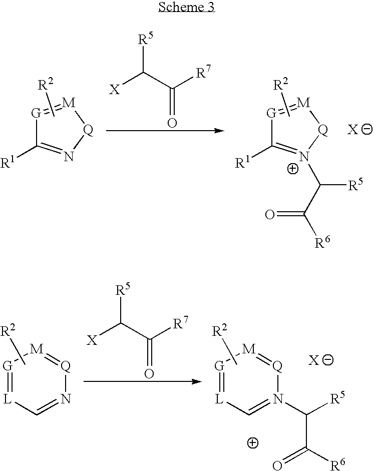

For example, a substituted 1,2,4-triazole (such as 4-phenyl substituted) can be alkylated with a substituted phenacyl bromide in acetone as shown in Scheme 4 (Surpateanu, G. G.; Vergoten, G.; Elass, A.; Surpateanu, G.; Heterocycles, 1999, 51, 2213-2220). Other 5-membered nitrogen heterocycles can be alkylated similarly.

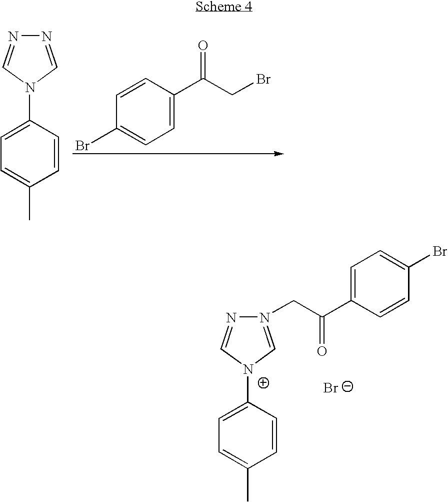

Analogously, a 1,2,4-triazole (which can be substituted) that is substituted at one ring nitrogen can be reacted with Y—X to form the charged species. For example, 1,2,4-triazole substituted at the 4 position (for example with amino, alkyl amino or alkyl) can be reacted with Y—X (for example 2-chloro-1-phenyl-ethanone).

In another example (1,3,4)-thiadiazole can be alkylated in acetonitrile with the same substituted phenacyl bromide to give 3-(4-bromophenacyl)-(1,3,4)-thiadiazolium bromide (Haug, E.; Kantlehner, W.; Hagen, H.; Speh, P.; Braeuner, H. Liebigs Ann. Chem., 1988, 605-608).

Six membered aromatic heterocycles such as pyridazine, pyrimidine, and pyridazine can be similarly alkylated with α-halo carbonyl containing reagents. For example, 1-(4-methylphenacyl)pyridazinium bromide can be prepared by reaction of equimolar amounts of 4-methylphenacyl bromide and pyridazine in refluxing acetonitrile (J. Med. Chem., 1989, 32, 2301-2306). Pyrimidines and can be prepared similarly. For example, 1-phenacylpyrimidinium bromide can be prepared by reaction of phenacyl bromide and pyrimidine (Chem. Ber., 1958, 91, 2832). Pyridine analogs, can also be prepared by this method. The ring N-atom of nicotinic acid benzyl ester can be alkylated, for instance, with 4-methoxyphenacyl bromide to provide 1-(4-methoxyphenyacyl)nicotinic acid benzyl ester bromide (Br. Patent 817103).

Compounds wherein Y is CH(R⁵)—C(O)—R⁷, wherein —C(O)—R⁷ comprises a carboxamide moiety, can be synthesized according to the method depicted in Scheme 5. An appropriately substituted amine can be condensed with an activated acetyl analog, containing a leaving group alpha to the carbonyl group (for example, an acid chloride such as α-chloroacetyl chloride), to provide a carboxamide. The carboxamide can then be used to alkylate the ring N atoms in the heterocycle to yield a compound of the invention. Alternatively, an activated acetyl analog with an α-halo leaving group can be used to directly alkylate the ring N-atom of the heterocycle. Displacement of the α-halo leaving group by an appropriately substituted amine also provides the N-alkylated heterocycle, wherein the —C(O)—R⁷ comprises a carboxamide.

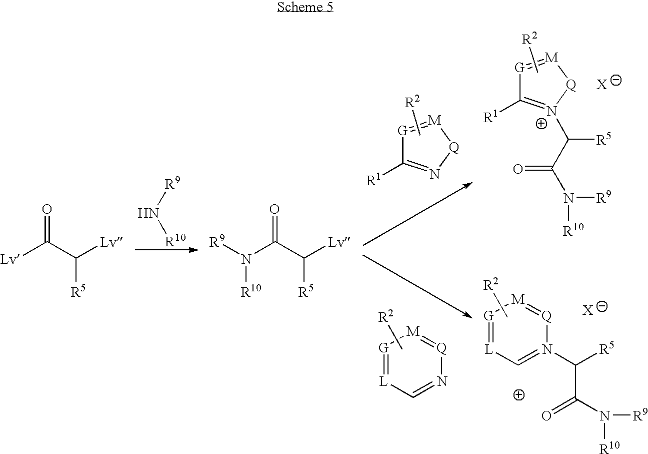

A useful synthetic route for the preparation of compounds of formula I wherein Y is —CH(R⁵)CN is shown in Scheme 6, wherein X is a halide, mesitylenesulfonate or other biologically acceptable anion. In Scheme 6, the appropriately substituted nitrogen containing aromatic heterocycle is contacted with an α halo substituted acetonitrile analog to produce cyanoalkyl substituted heterocycles. The reaction can be performed without any added solvent, or an anhydrous solvent can be utilized as the solvent medium. When a solvent is used, acetonitrile is a typical solvent for this reaction. Reaction times vary according to particular reactants and conditions, but are usually in the range of a few minutes to 48 hours at a temperature of 25-130° C.

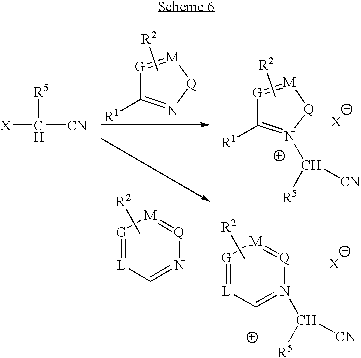

A synthetic scheme for making compounds of the formula I wherein Y is CH₂CH(OH)Rs is shown in Scheme 7. A hydroxyl is incorporated into a nucleophile used to derivatize a nitrogen heterocyclic compound, as follows: 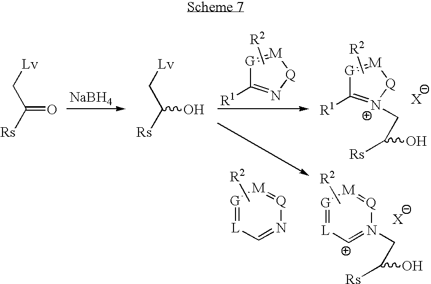
where Lv is a leaving group such as chloro. In a related synthesis, the carbonyl can be reduced with a stereoselective reducing agent such as (−) DIP-chloride [(−)-B-chlorodiisopinocampheylborane] or (+) DIP-chloride [(+)-B-chlorodiisopinocampheylborane] to provide specific stereoisomers of the alcohol. The alcohol can then be used to directly N-alkylate the heterocycle as above to prepare a compound of the invention enriched in the stereoisomer.

Compounds of the invention wherein R⁵ and R⁶ are both electron withdrawing groups such as ketones, carboxylic acids, carboxylic acid esters, carboxamides, or nitriles can be prepared as shown in Scheme 8. Suitable alkylating agents for compounds of this type include 2-halo substituted malonic acid derivatives such as 2-bromo diethyl malonate, 2-bromomalonamide, and the like. For instance, 1-bis(ethoxycarbonyl)methylpyridinium bromide can be prepared from the reaction of 2-bromo diethyl malonate and pyridine in refluxing acetone (J. Chem Soc., Perkin Trans. I, 1981, 3059). Likewise, 1-(2-malonamido)pyridinium bromide can be prepared from the reaction of 2-bromomalonamide and pyridine (U.S. Pat. No. 4,110,424).

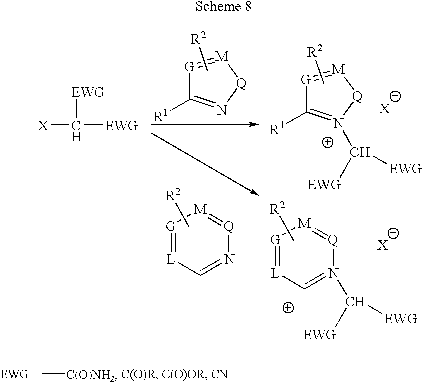

As is recognized in the art, many of the aromatic nitrogen heterocyclic analogs that serve as suitable precursors for the alkylation reactions discussed above are commercially available from chemical supply houses or are readily synthesized by methods well known in the art. For instance, certain substitution patterns can be obtained by electrophilic and nucleophilic substitution reactions on the heterocycle and are well known in the art. In addition, selected nitrogen heterocycles are susceptible to metallation with organoalkali reagents, for example, n-butyllithium. The intermediate lithio-heterocycles can be treated with electrophiles to provide additional routes to substituted aromatic nitrogen heterocyclic intermediates.

Certain aromatic nitrogen heterocyclic intermediates can be obtained by cyclization and cycloaddition reactions of substituted acyclic precursors that are well known in the art. Nonlimiting examples of the syntheses of nitrogen containing aromatic heterocyclic intermediates are described below.

Substituted pyrazoles can be obtained by reaction of 1,3-dicarbonyl compounds with hydrazines as was shown above in Scheme 2. As will be recognized by those in the art, use of unsymmetrically substituted 1,3-dicarbonyl compounds with alkyl or aryl hydrazines often lead to isomeric mixtures of pyrazole products. These isomeric mixtures can be separated by well-known separation techniques such as fractional crystallization, column chromatography, and the like. In addition, substituted pyrazole intermediates can be obtained by reaction of alkynyl carbonyls with hydrazines (Scheme 9) (Kost, A. N.; Grandberg Adv. Heterocyl. Chem., 1966, 6).

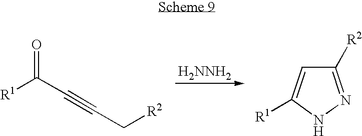

Substituted (1,2,3)triazole intermediates can be obtained by 1,3-dipolar cycloaddition reactions with activated alkynes (Scheme 10). For example, an alkyne diester can react with an azide to provide triazoles substituted at the 4 and 5 positions by ethoxycarbonyl groups, which serve as convenient moieties for further derivatization.

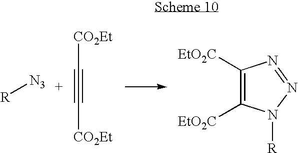

Benzotriazoles can be prepared, for example, by reaction of substituted ortho diaminobenzenes with nitrous acid (Scheme 11).

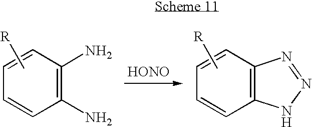

(1,2,4)Triazoles substituted in the 3 or 5 positions can be obtained from the condensation of acid hydrazides and thionoamides (Scheme 12). The triazole intermediates can be sequentially alkylated by two alkylating agents to provide compounds of the invention.

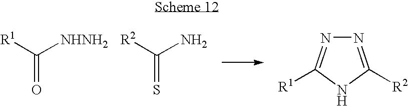

3- and 5-Aryl and alkyl isoxazoles can be prepared by reaction of the chloro substituted α,β-unsaturated ketones with hydroxylamine (Scheme 13). The isomeric products can be isolated by separation techniques such as fractional crystallization, distillation, or column chromatography. Alternatively, 5-aryl substituted isoxazoles can be prepared from acetophenones (Scheme 13, Lin, Y. Lang, S. A. J. Heterocyclic Chem., 1977, 14, 355).

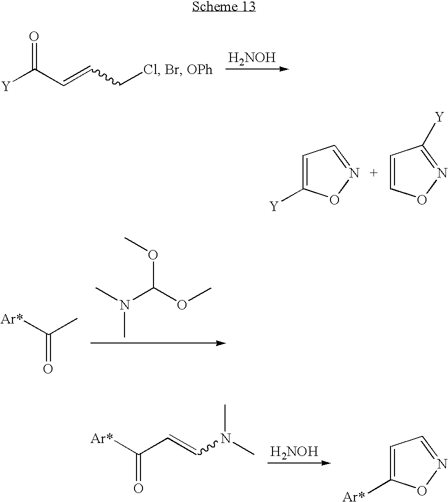

Alkyl and aryl substituted isothiazoles intermediates are prepared by the cyclization of β-imino thionocarbonyl compounds (Scheme 14). The cyclization is effected by oxidizing reagents well known in the art such as peroxides, chloranil, iodine, and the like. For example, starting material with an aryl thionocarbonyl group β-substituted to an imino group can be used to prepare a 5-aryl substituted isothiazole.

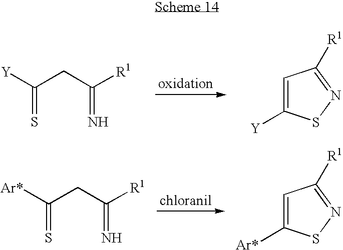

Suitable six-membered aromatic nitrogen heterocyclic intermediate such as pyrimidine, pyridazine, and pyridine can be obtained by ring cyclization and cycloaddition of substituted acyclic precursors as well. These heterocyclic intermediates serve as suitable substrates for the alkylation reactions discussed above to prepare the compounds of the invention.

Substituted pyrimidines can be obtained, for example, by the condensation of alkyl and aryl amidines with 1,3-dicarbonyl compounds (Scheme 15) or α,β,-unsaturated carbonyl compounds such as 3-ethoxymethacrolein.

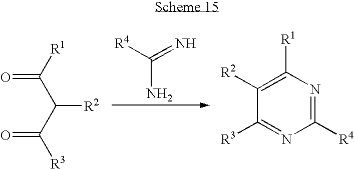

Benzo-fused pyrimidines (i.e., quinazolines) can be prepared, for instance, from benzene analogs containing an amino substituent ortho to a carbonyl (ketone or aldehyde) by acylation of the amino group with an alkanoyl or aroyl group, and then cyclization of the acylamino intermediate with ammonia (Scheme 16).

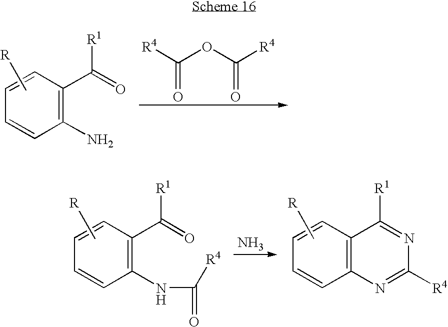

Pyridazines, useful as candidates for the alkylation reactions discussed above, can be prepared by reaction of hydrazine with 1,4-dicarbonyl compounds. The dihydro intermediates can be oxidized to give the desired pyridazines (Scheme 17). Phthalazines can be prepared in a similar fashion.

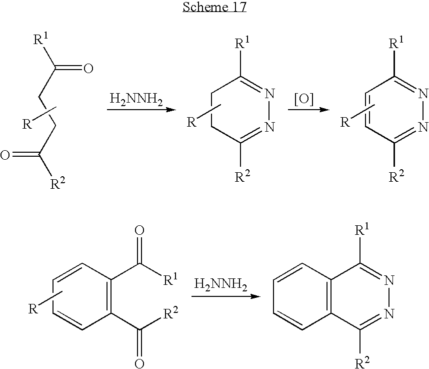

Cinnoline intermediates are prepared by cyclization of diazonium salts containing an ortho vinyl group (Scheme 18).

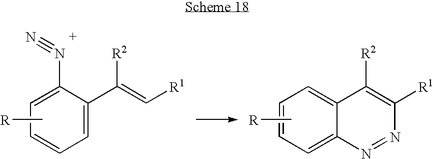

Quinolines that can serve as useful substrates for the alkylation reactions discussed above can be obtained from substituted benzene precursors by a number of methods known to those of ordinary skill in the art. For example, variations of the Skraup synthesis of quinolines can be used as shown in Scheme 19 (Jones, G., Quinolines, Wiley-Interscience, New York, 1977, p 93).

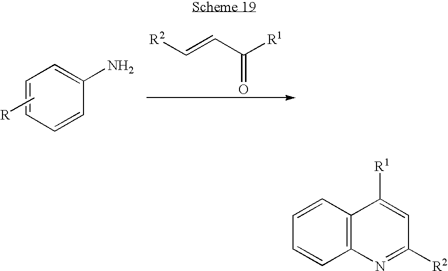

Substituted isoquinoline intermediates can be prepared by Bischler-Napieralski reaction followed by an oxidation step (Scheme 20).

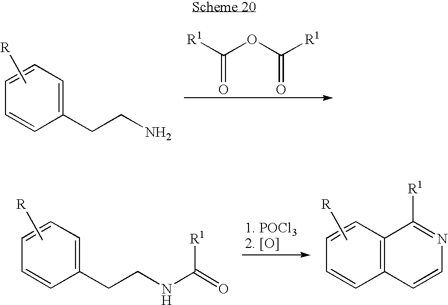

Pyridines, quinolines, and isoquinolines can be aminated with electrophilic aminating reagents such as hydroxylamine O-sulfonic acid (Scheme 21) or O-mesitylene sulfonylhydroxylamine.

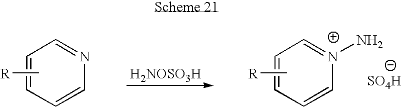

To treat glaucoma or reduced accommodation and associated symptoms, an effective amount of a pharmaceutical compound will be recognized by clinicians but includes an amount effective to treat, reduce, ameliorate, eliminate or prevent one or more symptoms of the disease sought to be treated or the condition sought to be avoided or treated, or to otherwise produce a clinically recognizable change in the pathology of the disease or condition.

In treating glaucoma, agents of the inventions can be administered concurrently or in a combined formulation with one or more α2-selective adrenergic agonists, carbonic anhydrase inhibitors or prostaglandin analogs. Examples of α2-selective adrenergic agonists include clonidine, apraclonidine, guanfacine, guanabenz and methyldopa, which are administered in effective amounts as is known in the art. Examples of carbonic anhydrase inhibitors include acetazolamide, dichlorphenamide and methazolamide, which are administered in effective amounts as is known in the art. Examples of prostaglandin analogs include PGE₂ and PGF_{2α} analogs, which are administered in effective amounts as is known in the art, including effective amounts administered by topical application to the eye. Thus, the invention further provides pharmaceutical compositions comprising an agent of the invention in combination with an effective amount of an α2-selective adrenergic agonist, carbonic anhydrase inhibitor, prostaglandin analog, or combination thereof.

Pharmaceutical compositions can be prepared to allow a therapeutically effective quantity of the compound of the present invention, and can include a pharmaceutically acceptable carrier, selected from known materials utilized for this purpose. See, e.g., Remington, The Science and Practice of Pharmacy, 1995; Handbook of Pharmaceutical Excipients, 3^{rd} Edition, 1999. Such compositions can be prepared in a variety of forms, depending on the method of administration.

In addition to the subject compound, the compositions of this invention can contain a pharmaceutically-acceptable carrier. The term “pharmaceutically-acceptable carrier”, as used herein, means one or more compatible solid or liquid filler diluents or encapsulating substances that are suitable for administration to an animal, including a mammal or human. The term “compatible”, as used herein, means that the components of the composition are capable of being commingled with the subject compound, and with each other, such that there is no interaction that would substantially reduce the pharmaceutical efficacy of the composition under ordinary use. Preferably when liquid dose forms are used, the compounds of the invention are soluble in the components of the composition. Pharmaceutically-acceptable carriers must, of course, be of sufficiently high purity and sufficiently low toxicity to render them suitable for administration to the animal being treated.

Some examples of substances which can serve as pharmaceutically-acceptable carriers or components thereof are sugars, such as lactose, glucose and sucrose; starches, such as corn starch and-potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with a blood-compatible suspending agent, the pH of which has been adjusted to about 7.4.

If the preferred mode of administering the subject compound is perorally, the preferred unit dosage form is therefore tablets, capsules, lozenges, chewable tablets, and the like. Such unit dosage forms comprise a safe and effective amount of the subject compound, which is preferably from about 0.7 or 3.5 mg to about 280 mg/70 kg, more preferably from about 0.5 or 10 mg to about 210 mg/70 kg. The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder-mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of this invention, and can be readily made by a person skilled in the art.

Peroral compositions also include liquid solutions, emulsions, suspensions, and the like. The pharmaceutically-acceptable carriers suitable for preparation of such compositions are well known in the art. Such liquid oral compositions preferably comprise from about 0.012% to about 0.933% of the subject compound, more preferably from about 0.033% to about 0.7%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. Peroral liquid compositions may also contain one or more components such as sweeteners, flavoring agents and colorants disclosed above.

Other compositions useful for attaining systemic delivery of the subject compounds include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Compositions can also be used to deliver the compound to the site where activity is desired; such as eye drops, gels and creams for ocular disorders.

Compositions of this invention include solutions or emulsions, preferably aqueous solutions or emulsions comprising a safe and effective amount of a subject compound intended for topical intranasal administration. Such compositions preferably comprise from about 0.01% to about 10.0% w/v of a subject compound, more preferably from about 0.1% to about 2.0%. Similar compositions are preferred for systemic delivery of subject compounds by the intranasal route. Compositions intended to deliver the compound systemically by intranasal dosing preferably comprise similar amounts of a subject compound as are determined to be safe and effective by peroral or parenteral administration. Such compositions used for intranasal dosing also typically include safe and effective amounts of preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acids and bases to adjust the pH of these aqueous compositions as needed. The compositions may also comprise local anesthetics or other actives. These compositions can be used as sprays, mists, drops, and the like.

Other preferred compositions of this invention include aqueous solutions, suspensions, and dry powders comprising a safe and effective amount of a subject compound intended for atomization and inhalation administration. Such compositions are typically contained in a container with attached atomizing means. Such compositions also typically include propellants such as chlorofluorocarbons 12/11 and 12/114, and more environmentally friendly fluorocarbons, or other nontoxic volatiles; solvents such as water, glycerol and ethanol, these include cosolvents as needed to solvate or suspend the active; stabilizers such as ascorbic acid, sodium metabisulfite; preservatives such as cetylpyridinium chloride and benzalkonium chloride; tonicity adjustors such as sodium chloride; buffers; and flavoring agents such as sodium saccharin. Such compositions are useful for treating respiratory disorders, such as asthma and the like.

Other preferred compositions of this invention include aqueous solutions comprising a safe and effective amount of a subject compound intended for topical intraocular administration. Such compositions preferably comprise from about 0.01% to about 0.8% w/v of a subject compound, more preferably from about 0.05% to about 0.3%. Such compositions also typically include one or more of preservatives, such as benzalkonium chloride or thimerosal, vehicles, such as poloxamers, modified celluloses, povidone and purified water; tonicity adjustors, such as sodium chloride, mannitol and glycerin; buffers such as acetate, citrate, phosphate and borate; antioxidants such as sodium metabisulfite, butylated hydroxy toluene and acetyl cysteine; acids and bases can be used to adjust the pH of these formulations as needed.

Other preferred compositions of this invention useful for peroral administration include solids, such as tablets and capsules, and liquids, such as solutions, suspensions and emulsions (preferably in soft gelatin capsules), comprising a safe and effective amount of a subject compound. Such compositions can be coated by conventional methods, typically with pH or time-dependent coatings, such that the subject compound is released in the gastrointestinal tract at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit™ coatings, waxes and shellac.

The compounds of the invention are administered by ocular, oral, parenteral, including, for example, using formulations suitable as eye drops. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. See, Remington's Pharmaceutical Sciences, 16th Ed., Mack Publishing, Easton, Pa., 1980, as well as later editions, for information on pharmaceutical compounding.

Numerous additional administration vehicles will be apparent to those of ordinary skill in the art, including without limitation slow release formulations, liposomal formulations and polymeric matrices.

In another preferred embodiment, the pharmaceutically effective amount is approximately 0.1 or 0.5 to 4 mg/kg body weight daily. Still more preferably, the pharmaceutically effective amount is approximately 1 mg/kg body weight daily. In a preferred embodiment, the amount is administered in once daily doses, each dose being approximately 1 mg/kg body weight.

Compounds of the invention can be used in conjunction with monitoring the improvement (decrease) in the intraocular pressure in a mammal using standard methodology.

The methods of the inventions can be assessed in animal models for ophthalmologic function. For example, improvements in fluid outflow facility can be studied in Rhesus monkeys treated with the compounds and methods of the invention. Aged Rhesus monkeys receive a single transcorneal injection of a test compound (compound of the invention) at a concentration of about 1 mM in the anterior chamber of one eye, and Barany's solution, as a control, in the adjacent eye. Needle outflow facility is measured under baseline and pilocarpine-stimulated conditions at time points (for example, 3, 8, 12 and 24 weeks), after the administration of the test compound. Increases in outflow facility in the drug treated vs. the control eye under baseline and cholinergic-stimulated (e.g. pilocarpine) conditions at the various time points are compared. As the enhancement of outflow facility can be influenced by the route of administration of the cholinergic agent, various routes of administration of the cholinergic agent can be used in the experiments. For instance, an intravenous administration versus a direct administration of pilocarpine can be compared. The above experiment demonstrates one method of measuring the improvement in ophthalmologic function. Such improvement has been illustrated with 4,5-dimethyl-3-(2-oxoethyl-phenethyl)thiazolium chloride, a compound believed to act by the same mechanism as those described here. See, U.S. application for “Methods for Treating Glaucoma I,” concurrently filed herewith.

In addition to measuring increased fluid outflow facility using the methods of the invention, improvements in pilocarpine-stimulated accommodation (i.e., the process of effecting refractive changes in the shape of the lens) can also be assessed in animal studies. As in the regulation of outflow facility, cholinergic input stimulates the movement of the ciliary muscle to control the shape of the lens, and allows accommodation in conditions of low illumination. Accommodation is impaired in a vast majority of individuals and begins to become noticeable to the individual around the age of 40 years. Interestingly, changes in accommodative response occur much earlier in life, around 18 years of age, and progresses until vision is noticeably impaired.

Physiological studies on accommodation are conducted following intraocular injection of a test compound and the results are compared relative to the results of control (untreated) animals. In the experiment, primates(for example, Rhesus monkeys) are treated twice a day for four days with 2 μg of prostaglandin F2α (PGF2α). On days 5-8 both eyes are treated first with 2 μg of PGF2α followed 2 hours later with an intraocular injection of 10 μL of the test compound of a final concentration of 1 mM. No injection is made to the control eye. 24 Hours after the last injection of the test compound, a course of therapy consisting of once a day dosing for a total of 4 days accommodative responses to i.m. pilocarpine administration is performed following phenylephrine refraction. Improvement in accommodation has been illustrated with 4,5-dimethyl-3-(2-oxoethyl-phenethyl)thiazolium chloride, a compound believed to act by the same mechanism as those described here. See, U.S. application for “Methods for Treating Glaucoma I,” concurrently filed herewith.

Compounds of the invention can be tested to determine corneal penetration to the anterior chamber of the eye following topical administration of eye drops. For example, a test compound is assayed in vitro through an intact rabbit cornea for transcorneal penetration in a standard diffusion chamber apparatus. Corneas are mounted in a chamber at 37° C. with the epithelial side exposed to the test compound in Barany's solution. 1.0 mL samples are taken from the endothelial side 1 hour after addition of the test compound at a final concentration of 1 mM to the epithelial chamber. The volume of the chamber is replaced with phosphate buffered saline. The amount of test compound can be measured using any means that can be used to separate the compound and measure its concentration. For example, an HPLC with an attached UV detector can be used to determine the concentration of the test compound that has penetrated the cornea. Penetration values are also determined at later time points, for example, at 5 hours.

Assessment of corneal penetration of compounds of the invention can be determined in vivo, for example, in Cynomolgus monkeys. During these studies, the penetration of a test compound is evaluated using an eye-cup which holds a solution of 10 mM of the test compound in Barany's solution for 5 hours. At the end of the experiment the eye cup is removed, the eye is repeatedly flooded with Barany's solution and a sample of intraocular fluid is removed from the anterior chamber with a needle inserted through the cornea. The quantity of the test compound in the intraocular fluid is determined using, for example, HPLC methods.

The activity of the compounds of the invention in breaking, reversing or inhibiting the formation of AGE's or AGE-mediated crosslinks can be assayed by any of the methods described in U.S. Pat. No. 5,853,703.

### EXAMPLE 1

### Cross-Linking Inhibition Assay

The following method was used to evaluate the ability of the compounds to inhibit the cross-linking of glycated bovine serum albumin (AGE-BSA) to rat tail tendon collagen-coated 96-well plates.

AGE-BSA was prepared by incubating BSA at a concentration of 200 mg per ml with 200 mM glucose in 0.4M sodium phosphate buffer, pH 7.4 at 37° C. for 12 weeks. The glycated BSA was then extensively dialyzed against phosphate buffer solution (PBS) for 48 hours with additional 5 times buffer exchanges. The rat tail tendon collagen coated plate was blocked first with 300 microliters of Superbloc blocking buffer (Pierce Chemical, Rockford, Ill.) for one hour. The blocking solution was removed from the wells by washing the plate twice with phosphate buffered saline (PBS)-Tween 20 solution (0.05% Tween 20) using a NUNC-multiprobe (Nalge Nunc, Rochester, N.Y.) or Dynatech ELISA-plate (Dynatech, Alexandria, Va.) washer. Cross-linking of AGE-BSA (1 to 10 microgram per well depending on the batch of AGE-BSA) to rat tail tendon collagen coated plate was performed with and without the testing compound dissolved in PBS buffer at pH 7.4 at one or more desired concentrations by the addition of 50 microliters each of the AGE-BSA diluted in PBS or in the solution of test compound at 37° C. for 4 hours. Unbrowned BSA in PBS buffer with or without testing compound were added to the separate wells as the blanks. The un-cross-linked AGE-BSA was then removed by washing the wells three times with PBS-Tween buffer. The amount of AGE-BSA crosslinked to the tail tendon collagen-coated plate was then quantitated using a polyclonal antibody raised against AGE-RNase. After a one-hour incubation period, AGE antibody was removed by washing 4 times with PBS-Tween.

The bound AGE antibody was then detected with the addition of horseradish peroxidase-conjugated secondary antibody—goat anti-rabbit immunoglobulin and incubation for 30 minutes. The substrate of 2,2-azino-di(3-ethylbenzthiazoline sulfonic acid) (ABTS chromogen) (Zymed Laboratories, Inc., South San Francisco, Calif.) was added. The reaction was allowed for an additional 15 minutes and the absorbance was read at 410 nm in a Dynatech plate reader.

### EXAMPLE 2

### Cross-Link Breaking Assay

To ascertain the ability of the compounds of the instant invention to break or reverse already formed advanced glycosylation endproducts, a sandwich enzyme immunoassay was applied. Generally, the assay utilizes collagen-coated 96 well microtiter plates that are obtained commercially. AGE-modified protein (AGE-BSA) is incubated on the collagen-coated wells for four hours, is washed off the wells with PBS-Tween and solutions of the test compounds are added. Following an incubation period of 16 hours (37° C.) cross-link-breaking is detected using an antibody raised against AGE-ribonuclease or with an antibody against BSA.

### Preparation of Solutions and Buffers

Bovine Serum Albumin (Type V) (BSA) (from Calbiochem) solution was prepared as follows: 400 mg of Type V BSA (bovine serum albumin) was added for each ml of 0.4 M sodium phosphate buffer, pH 7.4. A 400 mM glucose solution was prepared by dissolving 7.2 grams of dextrose in 100 ml of 0.4 M sodium phosphate buffer, pH 7.4. The BSA and glucose solutions were mixed 1:1 and incubated at 37° C. for 12 weeks. The pH of the incubation mixture was monitored weekly and adjusted to pH 7.4 if necessary. After 12 weeks, the AGE-BSA solution was dialyzed against PBS for 48 hours with four buffer changes, each at a 1:500 ratio of solution to dialysis buffer. Protein concentration was determined by the micro-Lowry method. The AGE-BSA stock solution was aliquoted and stored at −20° C.

Test compounds were dissolved in PBS and the pH was adjusted to pH 7.4, if necessary. AGE-BSA stock solution was diluted in PBS to measure maximum crosslinking and in the inhibitor solution for testing inhibitory activity of compounds. The concentration of AGE-BSA necessary to achieve the optimum sensitivity was determined by initial titration of each lot of AGE-BSA.

Substrates for detection of secondary antibody binding were prepared by diluting the HRP substrate buffer (Zymed) 1:10 in distilled water and mixing with ABTS chromogen (Zymed) 1:50 just prior to use.

### Assay Procedures

Biocoat plates were blocked with 300 microliters of Superbloc (Pierce Chemical). Plates were blocked for one hour at room temperature and were washed with PBS-Tween (0.05% v/v) three times with the Dynatech platewasher before addition of test reagents.

The first three wells of the Biocoat plate were used for the reagent blank. Fifty microliters of solutions AGE-BSA were added to test wells in triplicate and only PBS in blank wells. The plate was incubated at 37° C. for four hours and washed with PBS-Tween three times. Fifty microliters of PBS was added to the control wells and 50 microliters of the test prospective agent was added to the test wells and blank. The plate was incubated overnight (approximately 16 hours) with prospective agent, followed by washing in PBS before addition of primary antibody.

(Prior to use, each lot of primary antibody, either anti-BSA or anti-RNase, was tested for optimum binding capacity in this assay by preparing serial dilutions (1:500 to 1:2000) and plating 50 microliters of each dilution in the wells of Biocoat plates. Optimum primary antibody was determined from saturation kinetics.) Fifty microliters of primary antibody of appropriate dilution, was added and incubated for one hour at room temperature. The plate was then washed with PBS-Tween.

Plates were incubated with the secondary antibody, HRP-(Goat-anti-rabbit), which was diluted 1:4000 in PBS and used as the final secondary antibody. The incubation was performed at room temperature for thirty minutes.

Detection of maximum crosslinking and breaking of AGE crosslinking was performed as follows. HRP substrate (100 microliter) was added to each well of the plate and was incubated at 37° C. for fifteen minutes. Readings were taken in the Dynatech ELISA-plate reader.

Except where heteroaryl is separately recited for the same substituent, the term “heterocycle” includes heteroaryl.

Where noted above, publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

We claim:

1. A method of decreasing intraocular pressure or improving ocular accommodation in a subject in need thereof, the method comprising administering an intraocular pressure decreasing or accommodation improving amount of (A) a compound of formula (1):
Y—Ar⊕•X⁻
wherein:
a. Ar is a six membered heteroaryl ring having a first ring nitrogen and optionally second or third ring nitrogens, with the remaining ring atoms being carbon, oxygen, or sulfur;
b. Y is substituted on the first ring nitrogen;
c. Ar can be substituted on the ring carbon atoms
1. with one or more substituents independently selected from the group consisting ω-alkylenesulfonic acid, carbamoyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
2. two adjacent substitutions together with their ring carbons form a C₆- or C₁₀-aromatic fused ring system; or
3. two adjacent substitutions together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo; or
4. two adjacent substitutions together with their ring carbons form a fused five to eight membered heterocycle, wherein the ring fusion is at the carbon-carbon double bond of Ar, wherein the heterocycle consists of ring atoms selected from the group consisting of carbon, nitrogen, oxygen, and S(O)ₙ, wherein n=0, 1, or 2; or
5. two adjacent substitutions together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur;
d. Y is:
1. a group of the formula —CH(R⁵)-R⁶
(a) R⁵ is hydrogen, alkyl-, cycloalkyl-, alkenyl-, alkynyl-, hydroxyalkyl, aminoalkyl-, dialkylaminoalkyl-, (N-[C₆ or C₁₀]aryl)(N-alkyl)aminoalkyl-, piperidin-1-ylalkyl-, pyrrolidin-1-ylalkyl, azetidinylalkyl, 4-alkylpiperazine-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperazin-1-ylalkyl, piperidin-1-ylalkyl, [C₆ or C₁₀]aryl, or independently the same as R⁶;
(b) wherein R⁶ is
(1) hydrogen, alkyl, which may be substituted by alkoxycarbonyl, alkenyl, alkynyl, cyano-, cyanoalkyl-, or Rs, wherein Rs is a [C₆ or C₁₀]aryl or a heteroaryl containing 4-10 ring atoms of which 1-3 are heteroatoms selected from the group consisting of oxygen, nitrogen, and sulfur; or
(2) a group of the formula —W—R⁷, wherein R⁷ is alkyl, alkoxy, hydroxy, or Rs wherein W is —C(═O)— or —S(O)₂—;
(3) a group of the formula —W—OR⁸ wherein R⁸ is hydrogen or alkyl,
(4) a group of the formula —CH(OH)Rs; or
(5) a group of the formula —W—N(R⁹)R¹⁰, wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring, or
(iii) a heterocycle containing 4-10 ring atoms of which 1-3 are heteroatoms selected from the group consisting of oxygen, nitrogen and sulfur; or
(b) R⁹ is hydrogen or alkyl and R¹⁰ is Ar*; or
(c) R⁹ is hydrogen or alkyl, R¹⁰ is a heterocycle containing 4-10 ring atoms of which 1-3 are heteroatoms are selected from the group consisting of oxygen, nitrogen and sulfur; or
(d) R⁹ and R¹⁰ are both alkyl groups; or
(e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy; or
(f) R⁹ and R¹⁰ are both hydrogen; or
2. —NH₂, and
X is a pharmaceutically acceptable anion, which may be absent if the compound provides a neutralizing salt, or
a pharmaceutically acceptable salt of the compound,
wherein aryl, Ar or Ar* can be substituted with, in addition to any substitutions specifically noted, with one or more substituents selected from the group consisting of acylamino, acyloxyalkyl, alkanoyl, alkanoylalkyl, alkenyl, alkoxy, alkoxycarbonyl, alkoxycarbonaylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*C(O)NH—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl-, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl; and
wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylamino, alkylsulfonyl, alkylsulfinyl, alkylthio, amino, Ar*(CO)—, Ar*O—, Ar*—, carboxy, dialkylamino, fluoro, fluoroalkyl, difluoroalkyl, hydroxy, mercapto, sulfamoyl, or trifluoromethyl.

2. The method of claim 1, comprising administering an intraocular pressure decreasing or accommodation improving amount of a compound of formula I, wherein Y is according to formula —CH(R⁵)R⁶.

3. The method of claim 2, comprising administering an intraocular pressure decreasing or accommodation improving amount of a compound of formula I, wherein Y is according to formula —CH(R⁵)—W—R⁷.

4. The method of claim 2, comprising administering an intraocular pressure decreasing or accommodation improving amount of a compound of formula I, wherein Y is according to formula —CH(R⁵)—W—Rs.

5. The method of claim 1, comprising administering an intraocular pressure decreasing or accommodation improving amount of a compound of formula 1, wherein:
a. Ar can be substituted on ring carbon atoms
1. with one or more substituents independently selected from the group consisting of hydrogen, acylamino, alkanoyl, alkanoylalkyl, alkoxy, alkoxycarbonyl, alkoxycarbonylalkyl, alkyl, ω-alkylenesulfonic acid, carbamoyl, carboxy, carboxyalkyl, cycloalkyl, halo, hydroxy, (C₂-C₆)hydroxylalkyl, mercapto, nitro, sulfamoyl, sulfonic acid (—SO₃H), alkylsulfonyl (alkylSO₂—), alkylsulfinyl (alkylSO—) alkylthio, trifluoromethyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—, wherein Ar* may be substituted by one or more substituents as set forth above; or
2. two adjacent substituents together with their ring carbons form a C₆- or C₁₀-aromatic fused ring system; or
3. two adjacent substitutions together with their ring carbons form a fused C₅-C₇ fused cycloalkyl ring having no double bonds except the fused double bond of the Ar group, which cycloalkyl ring can be substituted by one or more of the group consisting of alkyl, amino, aminocarbonyl, carboxy, fluoro, or oxo, wherein multiple substituents are located on different carbon atoms of the cycloalkyl ring, except in the case of alkyl, and fluoro substituents, which can be located on the same or different carbon atoms;
b. Y is:
1. a group of the formula CH(R⁵)—R⁶
(a) R⁵ is hydrogen or alkyl;
(b) wherein R⁶ is
(1) hydrogen, alkyl, alkenyl, alkynyl, cyano, cyanoalkyl, or Rs, wherein Rs is a [C₆ or C₁₀]aryl or a heterocycle containing 4-10 ring atoms of which 1-3 are heteroatoms selected from the group consisting of oxygen, nitrogen and sulfur; or
(2) a group of the formula —W—R⁷, wherein R⁷ is alkyl, alkoxy, hydroxy, or Rs, wherein W is —C(═O)— or —S(O)₂—;
(3) a group of the formula —W—OR⁸ wherein R⁸ is hydrogen or alkyl,
(4) a group of the formula —CH(OH)Rs; or
(5) a group of the formula —W—N(R⁹)(R¹⁰), wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring containing at least one and up to three atoms of N for the 6-membered heteroaryl ring and from one to three atoms of N or one atom of O or S and zero to two atoms of N for the 5-membered heteroaryl ring; said heteroaryl ring can be optionally substituted with one or more halo or (C₁-C₃)alkenyledioxy groups, or fused to a phenyl ring, or
(b) R⁹ is hydrogen or alkyl and R¹⁰ is Ar*; or
(c) R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more halo or (C₁-C₃)alkylenedioxy; or
(d) R⁹ and R¹⁰ are both hydrogen; or
2. —NH₂, and
X is a pharmaceutically acceptable anion, which may be absent if the compound provides a neutralizing salt, or
a pharmaceutically acceptable salt of the compound,
wherein aryl, Ar or Ar* can be substituted with, in addition to any substitutions specifically noted, with one or more substituents selected from the group consisting of acylamino, acyloxyalkyl, alkanoyl, alkanoylalkyl, alkenyl, alkoxy, alkoxycarbonyl, alkoxycarbonylalkyl, alkyl, (C₁-C₃)alkylenedioxy, alkylsulfonyl, alkylsulfinyl, ω-alkylenesulfonic acid, alkylthio, allyl, Ar*C(O)—, Ar*C(O)NH—, Ar*O—, Ar*—, Ar*-alkyl-, carboxy, carboxyalkyl, cycloalkyl, halo, trifluoromethyl, hydroxy, (C₂-C₆)hydroxyalkyl, mercapto, nitro, sulfamoyl, sulfonic acid (SO₃H); and
wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylsulfonyl, alkylsufinyl, alkylthio, Ar*C(O)—, Ar*O—, Ar*—, carboxy, fluoro, fluoroalkyl, difluoroalkyl, hydroxy, mercapto, sulfamoyl, or trifluoromethyl, wherein multiple substituents are located on different atoms of the heterocyclic ring, with the proviso that alkyl, alkylcarbonyl, and fluoro substituents can be substituted on the same carbon atom of the heterocyclic ring.

6. The method of claim 5, comprising administering an intraocular pressure decreasing or accommodation improving amount of a compound of formula I, wherein Y is according to formula —CH(R⁵)—W—R⁶.

7. The method of claim 6, comprising administering an intraocular pressure decreasing or accommodation improving amount of a compound of formula I, wherein Y is according to formula —CH(R⁵)—W—R⁷.

8. The method of claim 6, comprising administering an intraocular pressure decreasing or accommodation improving amount of a compound of formula I, wherein Y is according to formula —CH(R⁵)—W—Rs.

9. The method of claim 1, wherein Y—Ar⊕•X⁻ is 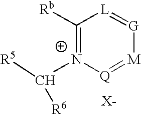
wherein L, G, M, Q, or R are independently N, C—R^{c}, C—R^{d}, C—R^{e}, C—R^{f},
wherein
1. R⁵ is H;
2. R⁶ is
(1) cyano or
(2) a group of the formula —W—R⁷, wherein R⁷ is alkyl or Rs, and W is C(═O)— or —S(═O)—;
3. R^{b}, R^{c}, R^{d}, and R^{e} are
(a) independently selected from the group consisting of hydrogen, acylamino, acyloxyalkyl, alkanoyl, alkanoylalkyl, alkenyl, alkoxy, alkoxycarbonyl, alkoxycarbonylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl-, 4-[C₆ or C₁₀]arylpiperazin-1-yl, 4-[C₆ or C₁₀]arylpiperdin-1-yl, azetidin-1-yl, and morpholin-4-yl, piperidin-1-yl;
(b) where any two of R^{b}, R^{c}, R^{d}, and R^{e} are adjacent, together with their ring carbons form a C₆- or C₁₀- aromatic fused ring system;
(c) where any two of R^{b}, R^{c}, R^{d}, and R^{e} are adjacent, together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo;
(d) wherein any two of R^{b}, R^{c}, R^{d}, and R^{e} are adjacent, together with their ring carbons form a fused five to eight membered heterocycle, wherein the ring fusion is at a carbon-carbon double bond of Ar, wherein the fused heterocycle consists of ring atoms selected from the group consisting of carbon, nitrogen, oxygen, and S(O)ₙ wherein n=0, 1, or 2;
(e) wherein any two of R^{b}, R^{c}, R^{d}, and R^{e} are adjacent, together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur, and wherein Ar has no more than three nitrogen atoms in the ring.
wherein Ar has no more than three nitrogen atoms in the ring.

10. The method of claim 1, wherein Ar is substituted on a said ring nitrogen with amino.

11. The method of claim 10, wherein Ar is further substituted with up to two aminos.

12. The method of claim 1, wherein said subject is a human.

13. A method of treating glaucoma in a subject in need thereof, the method comprising administering a pharmaceutically effective amount of (A) a compound of formula (I):
Y—Ar⊕•X⁻
wherein:
a. Ar is a six membered heteroaryl ring having a first ring nitrogen and optionally second or third ring nitrogens, with the remaining ring atoms being carbon, oxygen, or sulfur;
b. Y is substituted on the first ring nitrogen;
c. Ar can be substituted on the ring carbon atoms
1. with one or more substituents independently selected from the group consisting ω-alkylenesulfonic acid, carbamoyl, Ar*, Ar*-alkyl-, Ar*—O—, Ar*SO₂—, Ar*SO—, Ar*S—, Ar*SO₂NH—, Ar*NH, (N—Ar*)(N-alkyl)N—, Ar*C(O)—, Ar*C(O)NH—, Ar*NH—C(O)—, and (N—Ar*)(N-alkyl)N—C(O)—; or
2. two adjacent substitutions together with their ring carbons form a C₆- or C₁₀-aromatic fused ring system; or
3. two adjacent substitutions together with their ring carbons form a C₅-C₇ fused cycloalkyl ring having up to two double bonds including the fused double bond of the Ar group, which cycloalkyl ring can be substituted by one or more of the group consisting of alkyl, alkoxycarbonyl, amino, aminocarbonyl, carboxy, fluoro, or oxo; or
4. two adjacent substitutions together with their ring carbons form a fused five to eight membered heterocycle, wherein the ring fusion is at the carbon-carbon double bond of Ar, wherein the heterocycle consists of ring atoms selected from the group consisting of carbon, nitrogen, oxygen, and S(O)ₙ, wherein n=0, 1, or 2; or
5. two adjacent substitutions together with their ring carbons form a fused five or six membered heteroaryl ring, wherein the ring fusion is at a carbon-carbon double bond of Ar, wherein the fused heteroaryl ring consists of ring atoms selected from the group consisting of carbon, nitrogen, oxygen, and sulfur;
d. Y is:
1. a group of the formula —CH(R⁵)-R⁶
(a) R⁵ is hydrogen, alkyl-, cycloalkyl-, alkenyl-, alkynyl-, hydroxyalkyl, aminoalkyl-, dialkylaminoalkyl-, (N-[C₆ or C¹⁰]aryl)(N-alkyl)aminoalkyl-, piperidin-1-ylalkyl-, pyrrolidin-1-ylalkyl, azetidinylalkyl, 4-alkylpiperazine-1-ylalkyl, 4-alkylpiperidin-1-ylalkyl, 4-[C₆ or C₁₀]arylpiperazin-1-ylalkyl, 4-[C₆ or C₁₀]arylpiperidin-1-ylalkyl, azetidin-1-ylalkyl, morpholin-4-ylalkyl, thiomorpholin-4-ylalkyl, piperazin-1-ylalkyl, piperidin-1-ylalkyl, [C₆ or C₁₀]aryl, or independently the same as R⁶;
(b) wherein R⁶ is
(1) hydrogen, alkyl, which may be substituted by alkoxycarbonyl, alkenyl, alkynyl, cyano-, cyanoalkyl-, or Rs, wherein Rs is a [C₆ or C₁₀]aryl or a heteroaryl containing 4-10 ring atoms of which 1-3 are heteroatoms selected from the group consisting of oxygen, nitrogen, and sulfur; or
(2) a group of the formula —W—R⁷, wherein R⁷ is alkyl, alkoxy, hydroxy, or Rs wherein W is —C(═O)— or —S(O)₂—;
(3) a group of the formula —W—OR⁸ wherein R⁸ is hydrogen or alkyl,
(4) a group of the formula —CH(OH)Rs; or
(5) a group of the formula —W—N(R⁹)R¹⁰, wherein
(a) R⁹ is hydrogen and R¹⁰ is an alkyl or cycloalkyl, optionally substituted by
(i) [C₆ or C₁₀]aryl, or
(ii) a 5- or 6-membered heteroaryl ring, wherein the 6-membered heteroaryl ring contains at least one and up to three atoms of N and, the 5-membered heteroaryl ring contains from one to three atoms of N or one atom of O or S and zero to two atoms of N, said heteroaryl ring can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpholin-4-yl, piperidin-1-yl, halo or (C₁-C₃)alkylenedioxy groups, or fused to a phenyl or pyridine ring, wherein the ring fusion is at a carbon-carbon double bond of the heteroaryl ring, or
(iii) a heterocycle containing 4-10 ring atoms of which 1-3 are heteroatoms selected from the group consisting of oxygen, nitrogen and sulfur; or
(b) R⁹ is hydrogen or alkyl and R₁₀ is Ar*; or
(c) R⁹ is hydrogen or alkyl, R¹⁰ is a heterocycle containing 4-10 ring atoms of which 1-3 are heteroatoms are selected from the group consisting of oxygen, nitrogen and sulfur; or
(d) R⁹ and R¹⁰ are both alkyl groups; or
(e) R⁹ and R¹⁰ together with N form a heterocycle containing 4-10 ring atoms which can incorporate up to one additional heteroatom selected from the group of N, O or S in the ring, wherein the heterocycle is optionally substituted with (C₆-or C₁₀)aryl, (C₆-or C₁₀)arylalkyl, or a 5- or 6-membered heteroaryl ring containing at least one and up to three atoms of N for the 6-membered heteroaryl rings and from one to three atoms of N or one atom of O or S and zero to two atoms of N for the 5-membered heteroaryl rings, each such heteroaryl can be optionally substituted with one or more 1-pyrrolidinyl, 4-[C₆ or C₁₀]arylpiperazin-1-yl, 4-[C₆ or C₁₀]arylpiperidin-1-yl, azetidin-1-yl, morpholin-4-yl, thiomorpbolin-4-yl, piperidin-1-yl, halo or (C₁-C₃)alkylenedioxy; or
(f) R⁹ and R¹⁰ are both hydrogen; or
2. —NH₂, and
X is a pharmaceutically acceptable anion, which may be absent if the compound provides a neutralizing salt, or
a pharmaceutically acceptable salt of the compound,
wherein aryl, Ar or Ar* can be substituted with, in addition to any substitutions specifically noted, with one or more substituents selected from the group consisting of acylamino, acyloxyalkyl, alkanoyl, alkanoylalkyl, alkenyl, alkoxy, alkoxycarbonyl, alkoxycarbonaylalkyl, alkyl, alkylamino, (C₁-C₃)alkylenedioxy, alkylsulfonyl, alkylsulfinyl, ω-alkylenesulfonic acid, alkylthio, allyl, amino, Ar*C(O)—, Ar*C(O)NH—, Ar*O—, Ar*—, Ar*-alkyl-, carboxy, carboxyalkyl, cycloalkyl, dialkylamino, halo, trifluoromethyl, hydroxy, (C₂-C₆)hydroxyalkyl, mercapto, nitro, sulfamoyl, sulfonic acid, 1-pyrrolidinyl-, 4-[C₆ or C₁₀]arylpiperazin-1-yl, 4-[C₆ or C₁₀]arylpiperidin-1-yl, azetidin-1-yl, and morpholin-4-yl, thiomorpholin-4-yl, piperidin-1-yl; and
wherein heterocycles, except those of Ar or Ar*, can be substituted with, in addition to any substitutions specifically noted, acylamino, alkanoyl, alkoxy, alkoxycarbonyl, alkoxycarbonylalkyl, alkyl, alkylamino, alkylsulfonyl, alkylsulfinyl, alkylthio, amino, Ar*(CO)—, Ar*O—, Ar*—, carboxy, dialkylamino, fluoro, fluoroalkyl, difluoroalkyl, hydroxy, mercapto, sulfamoyl, or trifluoromethyl.
